(19) United States
(12) Patent Application Publication
(10) Pub. No.: US 2023/0410938 A1
(43) Pub. Date: Dec. 21, 2023

(54) PREDICTING PROTEIN STRUCTURES USING PROTEIN GRAPHS

(71) Applicant: DeepMind Technologies Limited, London (GB)

(72) Inventors: Alexander Pritzel, London (GB); Mikhail Figurnov, London (GB); John Jumper, London (GB)

(21) Appl. No.: 18/034,989

(22) PCT Filed: Nov. 23, 2021
(86) PCT No.: PCT/EP2021/082696

§ 371 Date: May 2, 2023

Related U.S. Application Data
(60) Provisional application No. 63/118,918, filed on Nov. 28, 2020.

(51) Int. Cl. G16B 15/20; G16B 15/30; G16B 30/10; G16B 40/20; G06N 3/08

(52) U.S. Cl. CPC G16B 15/20; G16B 15/30; G16B 30/10; G16B 40/20; G06N 3/08

(57) ABSTRACT

Methods, systems, and apparatus, including computer programs encoded on a computer storage medium, for determining a predicted structure of a protein. According to one aspect, there is provided a method comprising maintaining graph data representing a graph of the protein; obtaining a respective pair embedding for each edge in the graph; processing the pair embeddings using a sequence of update blocks, wherein each update block performs operations comprising, for each edge in the graph: generating a respective representation of each of a plurality of cycles in the graph that include the edge by, for each cycle, processing embeddings for edges in the cycle in accordance with the values of the update block parameters of the update block to generate the representation of the cycle; and updating the pair embedding for the edge using the representations of the cycles in the graph that include the edge.

### CROSS-REFERENCE TO RELATED APPLICATIONS

This application claims the benefit of the filing date of U.S. Provisional Patent Application Ser. No. 63/118,918, which was filed on Nov. 28, 2020, and which is incorporated herein by reference in its entirety.

### BACKGROUND

This specification relates to predicting protein structures.

A protein is specified by one or more sequences (“chains”) of amino acids. An amino acid is an organic compound which includes an amino functional group and a carboxyl functional group, as well as a side chain (i.e., group of atoms) that is specific to the amino acid. Protein folding refers to a physical process by which one or more sequences of amino acids fold into a three-dimensional (3-D) configuration. The structure of a protein defines the 3-D configuration of the atoms in the amino acid sequences of the protein after the protein undergoes protein folding. When in a sequence linked by peptide bonds, the amino acids may be referred to as amino acid residues.

Predictions can be made using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a protein structure prediction system implemented as computer programs on one or more computers in one or more locations for predicting protein structures using “protein graphs” (i.e., that represent proteins), “protein — multiple sequence alignment (MSA) graphs” (i.e., that jointly represent proteins and corresponding multiple sequence alignments (MSAs), or both.

Generally, a graph refers to a data structure that includes a set of nodes and a set of edges, where each edge connects a pair of nodes. An edge connecting pair of nodes in the graph can be a “directed” edge, i.e., that is associated with a direction that defines the edge as pointing from a “source” node to a “target” node, or an “undirected” edge, i.e., that connects a pair of nodes without being associated with a direction.

Each edge and each node in a graph can be associated with one or more embeddings, i.e., where an embedding for a node or an edge in the graph encodes information characterizing the node or edge.

The term “protein” can be understood to refer to any biological molecule that is specified by one or more sequences (or “chains”) of amino acids. For example, the term protein can refer to a protein domain, e.g., a portion of an amino acid chain of a protein that can undergo protein folding nearly independently of the rest of the protein. As another example, the term protein can refer to a protein complex, i.e., that includes multiple amino acid chains that jointly fold into a protein structure.

A “multiple sequence alignment” (MSA) for an amino acid sequence in a protein specifies a sequence alignment of the amino acid sequence with multiple additional amino acid sequences, referred to herein as “MSA sequences,” e.g., from other proteins, e.g., homologous proteins. More specifically, the MSA can define a correspondence between the positions in the amino acid chain and corresponding positions in multiple MSA sequences. A MSA for an amino acid sequence can be generated, e.g., by processing a database of amino acid sequences using any appropriate computational sequence alignment technique, e.g., progressive alignment construction. The MSA sequences can be understood as having an evolutionary relationship, e.g., where each MSA sequence may share a common ancestor. The correlations between the amino acids in the MSA sequences for an amino acid chain can encode information that is relevant to predicting the structure of the amino acid chain.

An “embedding” of an entity (e.g., a pair of amino acids) can refer to a representation of the entity as an ordered collection of numerical values, e.g., a vector or matrix of numerical values.

The structure of a protein can be defined by a set of structure parameters. A set of structure parameters defining the structure of a protein can be represented as an ordered collection of numerical values. A few examples of possible structure parameters for defining the structure of a protein are described in more detail next.

In one example, the structure parameters defining the structure of a protein include: (i) location parameters, and (ii) rotation parameters, for each amino acid in the protein.

The location parameters for an amino acid can specify a predicted 3-D spatial location of a specified atom in the amino acid in the structure of the protein. The specified atom can be the alpha carbon atom in the amino acid, i.e., the carbon atom in the amino acid to which the amino functional group, the carboxyl functional group, and the side chain are bonded. The location parameters for an amino acid can be represented in any appropriate coordinate system, e.g., a three-dimensional [x, y, z] Cartesian coordinate system.

The rotation parameters for an amino acid can specify the predicted “orientation” of the amino acid in the structure of the protein. More specifically, the rotation parameters can specify a 3-D spatial rotation operation that, if applied to the coordinate system of the location parameters, causes the three “main chain” atoms in the amino acid to assume fixed positions relative to the rotated coordinate system. The three main chain atoms in the amino acid can refer to the linked series of nitrogen, alpha carbon, and carbonyl carbon atoms in the amino acid. The rotation parameters for an amino acid can be represented, e.g., as an orthonormal 3×3 matrix with determinant equal to 1.

Generally, the location and rotation parameters for an amino acid define an egocentric reference frame for the amino acid. In this reference frame, the side chain for each amino acid may start at the origin, and the first bond along the side chain (i.e., the alpha carbon—beta carbon bond) may be along a defined direction.

In another example, the structure parameters defining the structure of a protein can include a “distance map” that characterizes a respective estimated distance (e.g., measured in angstroms) between each pair of amino acids in the protein. A distance map can characterize the estimated distance between a pair of amino acids, e.g., by a probability distribution over a set of possible distances between the pair of amino acids.

In another example, the structure parameters defining the structure of a protein can define a three-dimensional (3D) spatial location of each atom in each amino acid in the structure of the protein.

The protein structure prediction system described herein can be used to obtain a ligand such as a drug or a ligand of an industrial enzyme. For example, a method of obtaining a ligand may include obtaining a target amino acid sequence, in particular the amino acid sequence of a target protein, e.g. a drug target, and processing an input based on the target amino acid sequence using the protein structure prediction system to determine a (tertiary) structure of the target protein, i.e., the predicted protein structure. The method may then include evaluating an interaction of one or more candidate ligands with the structure of the target protein. The method may further include selecting one or more of the candidate ligands as the ligand dependent on a result of the evaluating of the interaction.

In some implementations, evaluating the interaction may include evaluating binding of the candidate ligand with the structure of the target protein. For example, evaluating the interaction may include identifying a ligand that binds with sufficient affinity for a biological effect. In some other implementations, evaluating the interaction may include evaluating an association of the candidate ligand with the structure of the target protein which has an effect on a function of the target protein, e.g., an enzyme. The evaluating may include evaluating an affinity between the candidate ligand and the structure of the target protein, or evaluating a selectivity of the interaction. The candidate ligand(s) may be selected according to which have the highest affinity.

The candidate ligand(s) may be derived from a database of candidate ligands, and/or may be derived by modifying ligands in a database of candidate ligands, e.g., by modifying a structure or amino acid sequence of a candidate ligand, and/or may be derived by stepwise or iterative assembly/optimization of a candidate ligand.

The evaluation of the interaction of a candidate ligand with the structure of the target protein may be performed using a computer-aided approach in which graphical models of the candidate ligand and target protein structure are displayed for user-manipulation, and/or the evaluation may be performed partially or completely automatically, for example using standard molecular (protein-ligand) docking software. In some implementations the evaluation may include determining an interaction score for the candidate ligand, where the interaction score includes a measure of an interaction between the candidate ligand and the target protein. The interaction score may be dependent upon a strength and/or specificity of the interaction, e.g., a score dependent on binding free energy. A candidate ligand may be selected dependent upon its score.

In some implementations the target protein includes a receptor or enzyme and the ligand is an agonist or antagonist of the receptor or enzyme. In some implementations the method may be used to identify the structure of a cell surface marker. This may then be used to identify a ligand, e.g., an antibody or a label such as a fluorescent label, which binds to the cell surface marker. This may be used to identify and/or treat cancerous cells.

In some implementations the ligand is a drug and the predicted structure of each of a plurality of target proteins is determined, and the interaction of the one or more candidate ligands with the predicted structure of each of the target proteins is evaluated. Then one or more of the candidate ligands may be selected either to obtain a ligand that (functionally) interacts with each of the target proteins, or to obtain a ligand that (functionally) interacts with only one of the target proteins. For example in some implementations it may be desirable to obtain a drug that is effective against multiple drug targets. Also or instead it may be desirable to screen a drug for off-target effects. For example in agriculture it can be useful to determine that a drug designed for use with one plant species does not interact with another, different plant species and/or an animal species.

In some implementations the candidate ligand(s) may include small molecule ligands, e.g., organic compounds with a molecular weight of <900 daltons. In some other implementations the candidate ligand(s) may include polypeptide ligands, i.e., defined by an amino acid sequence.

In some cases, the protein structure prediction system can be used to determine the structure of a candidate polypeptide ligand, e.g., a drug or a ligand of an industrial enzyme. The interaction of this with a target protein structure may then be evaluated; the target protein structure may have been determined using a structure prediction neural network or using conventional physical investigation techniques such as x-ray crystallography and/or magnetic resonance techniques or cryogenic electron microscopy.

In another aspect there is provided a method of using a protein structure prediction system to obtain a polypeptide ligand (e.g., the molecule or its sequence). The method may include obtaining an amino acid sequence of one or more candidate polypeptide ligands. The method may further include using the protein structure prediction system to determine (tertiary) structures of the candidate polypeptide ligands. The method may further include obtaining a target protein structure of a target protein, in silico and/or by physical investigation, and evaluating an interaction between the structure of each of the one or more candidate polypeptide ligands and the target protein structure. The method may further include selecting one or more of the candidate polypeptide ligands as the polypeptide ligand dependent on a result of the evaluation.

As before evaluating the interaction may include evaluating binding of the candidate polypeptide ligand with the structure of the target protein, e.g., identifying a ligand that binds with sufficient affinity for a biological effect, and/or evaluating an association of the candidate polypeptide ligand with the structure of the target protein which has an effect on a function of the target protein, e.g., an enzyme, and/or evaluating an affinity between the candidate polypeptide ligand and the structure of the target protein, or evaluating a selectivity of the interaction. In some implementations the polypeptide ligand may be an aptamer. Again the polypeptide candidate ligand(s) may be selected according to which have the highest affinity.

As before the selected polypeptide ligand may comprise a receptor or enzyme and the ligand may be an agonist or antagonist of the receptor or enzyme. In some implementations the polypeptide ligand may comprises an antibody and the target protein comprises an antibody target, for example a virus, in particular a virus coat protein, or a protein expressed on a cancer cell. In these implementations the antibody binds to the antibody target to provide a therapeutic effect. For example, the antibody may bind to the target and act as an agonist for a particular receptor; alternatively, the antibody may prevent binding of another ligand to the target, and hence prevent activation of a relevant biological pathway.

Implementations of the method may further include synthesizing, i.e., making, the small molecule or polypeptide ligand. The ligand may be synthesized by any conventional chemical techniques and/or may already be available, e.g., may be from a compound library or may have been synthesized using combinatorial chemistry.

The method may further include testing the ligand for biological activity in vitro and/or in vivo. For example the ligand may be tested for ADME (absorption, distribution, metabolism, excretion) and/or toxicological properties, to screen out unsuitable ligands. The testing may include, e.g., bringing the candidate small molecule or polypeptide ligand into contact with the target protein and measuring a change in expression or activity of the protein.

In some implementations a candidate (polypeptide) ligand may include: an isolated antibody, a fragment of an isolated antibody, a single variable domain antibody, a bi- or multi-specific antibody, a multivalent antibody, a dual variable domain antibody, an immuno-conjugate, a fibronectin molecule, an adnectin, an DARPin, an avimer, an affibody, an anticalin, an affilin, a protein epitope mimetic or combinations thereof. A candidate (polypeptide) ligand may include an antibody with a mutated or chemically modified amino acid Fc region, e.g., which prevents or decreases ADCC (antibody-dependent cellular cytotoxicity) activity and/or increases half-life when compared with a wild type Fc region. Candidate (polypeptide) ligands may include antibodies with different CDRs (Complementarity-Determining Regions).

The protein structure prediction system described herein can also be used to obtain a diagnostic antibody marker of a disease. There is also provided a method that, for each of one or more candidate antibodies e.g. as described above, uses the protein structure prediction system to determine a predicted structure of the candidate antibody. The method may also involve obtaining a target protein structure of a target protein, evaluating an interaction between the predicted structure of each of the one or more candidate antibodies and the target protein structure, and selecting one of the one or more of the candidate antibodies as the diagnostic antibody marker dependent on a result of the evaluating, e.g. selecting one or more candidate antibodies that have the highest affinity to the target protein structure. The method may include making the diagnostic antibody marker. The diagnostic antibody marker may be used to diagnose a disease by detecting whether it binds to the target protein in a sample obtained from a patient, e.g. a sample of bodily fluid. As described above, a corresponding technique can be used to obtain a therapeutic antibody (polypeptide ligand).

Misfolded proteins are associated with a number of diseases. Thus in a further aspect there is provided a method of using the protein structure prediction system to identify the presence of a protein mis-folding disease. The method may include obtaining an amino acid sequence of a protein and using the protein structure prediction system to determine a structure of the protein. The method may further include obtaining a structure of a version of the protein obtained from a human or animal body, e.g., by conventional (physical) methods. The method may then include comparing the structure of the protein with the structure of the version obtained from the body and identifying the presence of a protein mis-folding disease dependent upon a result of the comparison. That is, mis-folding of the version of the protein from the body may be determined by comparison with the in silico determined structure.

In general identifying the presence of a protein mis-folding disease may involve obtaining an amino acid sequence of a protein, using an amino acid sequence of the protein to determine a structure of the protein, as described herein, and comparing the structure of the protein with the structure of a baseline version of the protein, identifying the presence of a protein mis-folding disease dependent upon a result of the comparison. For example the compared structures may be those of a mutant and wild-type protein. In implementations the wild-type protein may be used as the baseline version but in principle either may be used as the baseline version.

In some other aspects a computer-implemented method as described above or herein may be used to identify active/binding/blocking sites on a target protein from its amino acid sequence.

According to one aspect there is provided a method performed by one or more data processing apparatus for determining a predicted structure of a protein, the method comprising: maintaining graph data representing a graph of the protein, wherein the graph comprises a set of nodes and a set of edges, wherein the set of nodes comprises a plurality of amino acid nodes that each represent a respective amino acid in the protein, and wherein the set of edges comprises a respective edge connecting each pair of amino acid nodes in graph; obtaining a respective pair embedding for each edge in the graph that connects a pair of amino acid nodes; processing an input comprising the pair embeddings using an embedding neural network, wherein the embedding neural network comprises a sequence of update blocks and uses the update blocks to repeatedly update the pair embeddings, wherein each update block has a plurality of update block parameters and performs operations comprising: receiving the pair embeddings; updating the pair embeddings in accordance with values of the update block parameters of the update block, comprising, for each edge in the graph that connects a pair of amino acid nodes: generating a respective representation of each of a plurality of cycles in the graph that include the edge by, for each cycle, processing embeddings for edges in the cycle in accordance with the values of the update block parameters of the update block to generate the representation of the cycle; and updating the pair embedding for the edge using the representations of the cycles in the graph that include the edge; and after processing the pair embeddings using the embedding neural network, determining the predicted structure of the protein based on the pair embeddings.

In some implementations, generating a respective representation of each of a plurality of cycles in the graph that include the edge comprises generating a respective representation of every cycle in the graph that includes the edge and that has a predefined length.

In some implementations, the predefined length is three.

In some implementations, updating the pair embedding for the edge using the representations of the cycles in the graph that include the edge comprises: processing the pair embedding for the edge and the representations of the cycles in the graph that include the edge, in accordance with the values of the update block parameters of the update block, to generate a residual embedding; and adding the residual embedding to the pair embedding for the edge.

In some implementations, processing the pair embedding for the edge and the representations of the cycles in the graph that include the edge, in accordance with the values of the update block parameters of the update block, to generate a residual embedding comprises: summing the representations of the cycles in the graph that include the edge; and processing the pair embedding for the edge and the sum of the representations of the cycles in the graph that include the edge using one or more neural network layers to generate the residual embedding.

In some implementations, each update block receives a multiple sequence alignment (MSA) representation for the protein that represents a respective MSA corresponding to each amino acid chain in the protein; and wherein the pair embedding for each edge is updated based at least in part on the MSA representation.

In some implementations, the set of nodes of the graph comprises a plurality of multiple sequence alignment (MSA) sequence nodes that each represent a respective MSA sequence corresponding to an amino acid chain in the protein, the set of edges of the graph comprises a respective edge connecting each MSA sequence node in the graph to each amino acid node in the graph, and the MSA representation for the protein comprises a respective embedding for each edge in the graph that connects a MSA sequence node to an amino acid node.

In some implementations, for each edge in the graph that connects a pair of amino acid nodes, generating the respective representation of each of the plurality of cycles in the graph that include the edge comprises generating a respective representation of each of one or more cycles in the graph that include an edge that connects a MSA sequence node to an amino acid node.

In some implementations, each update block further performs operations comprising: applying a transformation operation to the MSA representation; and updating the pair embeddings by adding a result of the transformation operation to the pair embeddings.

In some implementations, the transformation operation comprises an outer product mean operation.

In some implementations, each update block further performs operations comprising: updating the MSA representation based on the pair embeddings.

In some implementations, updating the MSA representation based on the pair embeddings comprises: updating the MSA representation using attention over embeddings in the MSA representation, wherein the attention is conditioned on the pair embeddings.

In some implementations, updating the MSA representation using attention over the embeddings in the MSA representation comprises: generating, based on the MSA representation, a plurality of attention weights; generating, based on the pair embeddings, a respective attention bias corresponding to each of the attention weights; generating a plurality of biased attention weights based on the attention weights and the attention biases; and updating the embeddings in the MSA representation using attention over the embeddings in the MSA representation based on the biased attention weights.

In some implementations, updating the embeddings in the MSA representation using attention based on the biased attention weights comprises, for each embedding in the MSA representation: updating the embedding, based on the biased attention weights, using attention over only embeddings in the MSA representation that are located in a same row as the embedding in an arrangement of the embeddings in the MSA representation into a two-dimensional array.

Particular embodiments of the subject matter described in this specification can be implemented so as to realize one or more of the following advantages.

This specification describes a protein structure prediction system that can predict the structure of a protein using a protein graph that represents the protein. The protein graph can include a respective node representing each amino acid in the protein, and edges between nodes in the protein graph can be associated with “pair” embeddings. A pair embedding associated with an edge that connects a pair of nodes in the protein graph encodes information that characterizes the relationship between a corresponding pair of amino acids in the protein, e.g., the distance between the pair of amino acids in the protein structure.

As part of predicting the protein structure, the protein structure prediction system enriches the information content of the pair embeddings associated with the edges of the protein graph by processing data representing the protein graph using a graph neural network. The graph neural network can enrich the information content of the pair embeddings by sharing information among pair embeddings associated with edges that form cycles in the protein graph. Spatial distances between pairs of amino acids in a protein structure have a transitive relationship, and updating the pair embeddings along cycles in the protein graph enables the graph neural network to exploit this transitive relationship to effectively enrich the pair embeddings, as will be described in more detail below.

After enriching the pair embeddings associated with the edges in the protein graph, the protein structure prediction system processes the pair embeddings to predict the protein structure. Using a graph neural network to enrich the information content of the pair embeddings associated with the edges in the protein graph can enable the structure prediction system to predict protein structures with high accuracy.

This protein structure prediction system can also predict the structure of a protein using a protein—multiple sequence alignment (MSA) graph that jointly represents the protein and MSAs corresponding to the amino acid chains of the protein. As part of predicting the protein structure, the protein structure prediction system enriches the information content of embeddings associated with edges in the protein-MSA graph by processing data representing the protein-MSA graph using a graph neural network.

After enriching the embeddings associated with the edges in the protein-MSA graph, the protein structure prediction system processes the edge embeddings of the protein-MSA graph to predict the protein structure. By representing both the protein and the MSAs corresponding the protein in one graph, and propagating information through the graph to enrich the embeddings associated with the edges in the graph, the protein structure prediction system can generate edge embeddings that enable protein structures to be predicted with high accuracy.

The systems described in this specification can predict the structure of a protein by a single forward pass through a collection of jointly trained neural networks, which may take less than one second. In contrast, some conventional systems predict the structure of a protein by an extended search process through the space of possible protein structures to optimize a scalar score function, e.g., using simulated annealing or gradient descent techniques. Such a search process may require millions of search iterations and consume hundreds of central processing unit (CPU) hours. Predicting protein structures by a single forward pass through a collection of neural networks may enable the systems described in this specification to consume fewer computational resources (e.g., memory and computing power) than systems that predict protein structures by an iterative search process.

The structure of a protein determines the biological function of the protein. Therefore, determining protein structures may facilitate understanding life processes (e.g., including the mechanisms of many diseases) and designing proteins (e.g., as drugs, or as enzymes for industrial processes). For example, which molecules (e.g., drugs) will bind to a protein (and where the binding will occur) depends on the structure of the protein. Since the effectiveness of drugs can be influenced by the degree to which they bind to proteins (e.g., in the blood), determining the structures of different proteins may be an important aspect of drug development. However, determining protein structures using physical experiments (e.g., by x-ray crystallography) can be time-consuming and very expensive. Therefore, the protein prediction systems described in this specification may facilitate areas of biochemical research and engineering which involve proteins (e.g., drug development).

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows a protein graph representing a protein.

FIG. 2 shows a protein-MSA graph that jointly represents a protein and MSAs corresponding to the amino acid chains of the protein.

FIG. 3 shows an example protein structure prediction system that, as part of predicting the structure of a protein, uses graph neural networks to process protein graphs and/or protein-MSA graphs for the protein.

FIG. 4 shows an example architecture of an embedding neural network that is configured to process the MSA representation and the pair embeddings to generate the updated MSA representation and the updated pair embeddings.

FIG. 5 shows a possible architecture of an update block of the embedding neural network.

FIG. 6 shows an example architecture of a MSA update block.

FIG. 7A shows an example architecture of a pair update block that updates the current pair embeddings by processing a protein graph for the protein using a graph neural network.

FIG. 7B shows an example architecture of a pair update block that updates the current pair embeddings by processing a protein-MSA graph for the protein using a graph neural network.

FIG. 8 shows an example architecture of a folding neural network that generates a set of structure parameters that define the predicted protein structure.

FIG. 9 illustrates the torsion angles between the bonds in the amino acid.

FIG. 10 is an illustration of an unfolded protein and a folded protein.

FIG. 11 is a flow diagram of an example process determining a predicted structure of a protein.

Like reference numbers and designations in the various drawings indicate like elements.

### DETAILED DESCRIPTION

This specification describes a protein structure prediction system (“system”) that is configured to process data defining one or more amino acid chains of a protein to generate a set of structure parameters that define a predicted protein structure, i.e., a prediction of the structure of the protein.

To generate the structure parameters defining the predicted protein structure, the system initializes: (i) a set of “pair” embeddings for the protein, and in implementations though not essentially, (ii) a multiple sequence alignment (MSA) representation for the protein.

The set of pair embeddings for the protein includes a respective pair embedding corresponding to each pair of amino acids in the protein. The pair embeddings for the protein can be represented as an N×N array of embeddings (i.e., a 2-D array of embeddings having N rows and N columns), where N is the number of amino acids in the protein. The amino acids in the protein can be indexed by the set {1, . . . , N}, and the pair embedding at position (i,j) in the N×N array of pair embeddings can be the pair embedding associated with pair of amino acids composed of amino acid i and amino acid j. For convenience, the pair embedding at position (i,j) in the N×N array of pair embedding may be referred to as the pair embedding corresponding to the amino acid pair (i,j). Generally, the pair embeddings can encode information about the inter-relationships between the amino acids in the protein. For example, the pair embedding for a pair of amino acids can encode protein structural information, e.g., that characterizes the spatial distance between the pair of amino acids in the protein structure.

The MSA representation for the protein can be represented as an L×N array of embeddings (i.e., a 2-D array of embeddings having L rows and N columns), where N is the number of amino acids in the protein. Each row of the MSA representation corresponds to a MSA sequence from an MSA corresponding to an amino acid chain in the protein. The MSA sequences associated with the protein can be indexed by the set {1, . . . , L} and the amino acids in the protein can be indexed by the set {1, . . . , N}. For convenience, the embedding at position (i,j) in the L×N array of embeddings of the MSA representation may be referred to as the embedding corresponding to MSA sequence—amino acid pair (i,j). Generally, the MSA representation can encode information about the correlations between the identities of the amino acids in different positions among a set of evolutionarily-related amino acid chains.

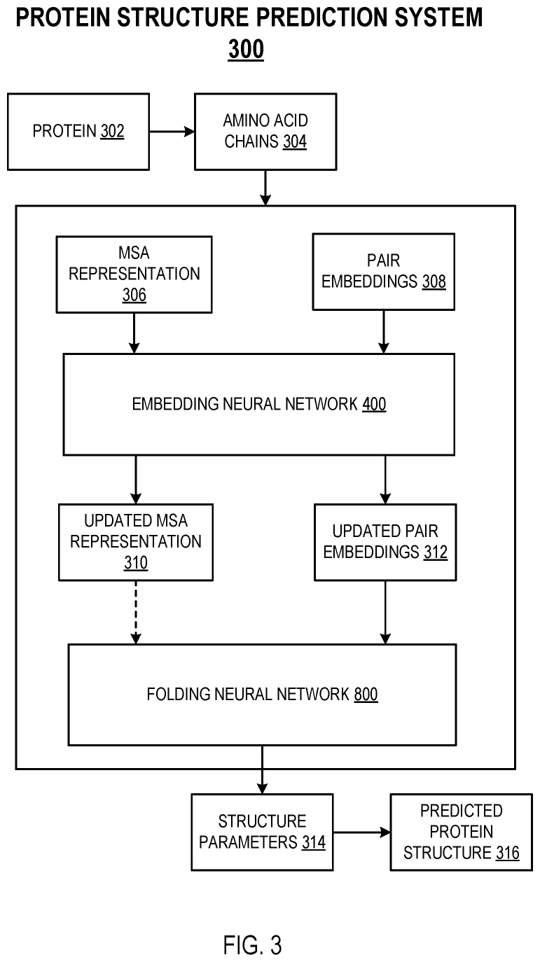

The system can initialize the pair embeddings and the MSA representation in a variety of ways, as will be described in more detail below, e.g., with reference to FIG. 3.

After initializing the pair embeddings and the MSA representation, the system repeatedly updates the pair embeddings and the MSA representation as part of predicting the structure of the protein. The system can use a neural network referred to herein as a “graph neural network” to update the pair embeddings, the MSA representation, or both, as will be described in more detail below.

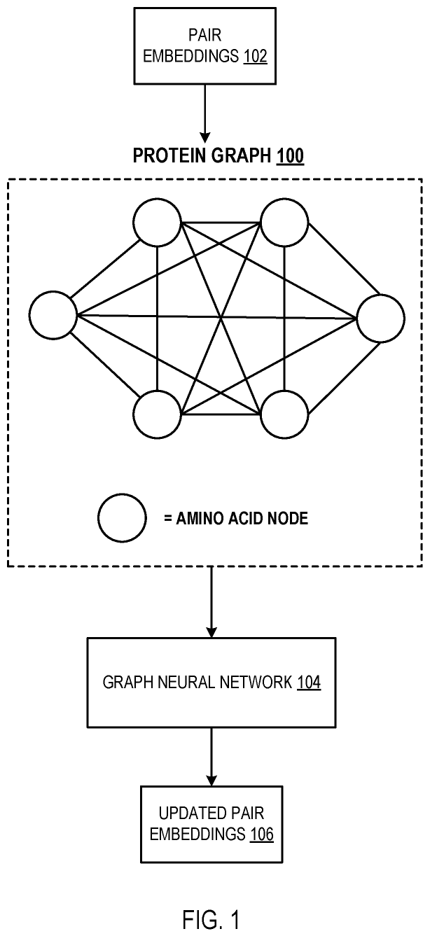

FIG. 1 shows a protein graph 100 representing a protein. The protein graph 100 includes a respective node representing each amino acid in the protein. For convenience, the nodes in the protein graph 100 may be referred to herein as “amino acid (AA) nodes” or “AA nodes.” Thus there may be an AA node for each (successive) amino acid in one or more sequences of amino acids that specify the protein.

The protein graph 100 can be a “fully connected” graph, i.e., that includes a respective edge between each pair of AA nodes. As an illustrative example, in the (simplified) protein graph shown in FIG. 1, each of the AA nodes is connected to each of the other AA nodes.

In some implementations, the edges between pairs of AA nodes in the protein graph 100 are directed edges. That is, for every possible pair of AA nodes, the protein graph 100 can include a directed edge pointing from the first AA node in the pair to the second AA node in the pair, and a directed edge pointing from the second AA node to the first AA node. In some implementations, the edges between pairs of AA nodes are undirected edges.

The set of pair embeddings for the protein can be associated with the edges in the protein graph, i.e., such that pair embedding for amino acid pair (i,j) is associated with the edge pointing from the AA node representing amino acid i to the AA node representing amino acid j.

The system can provide data representing the protein graph 100 (i.e., including the pair embeddings 102 associated with the edges in the protein graph 100) to a graph neural network 104 that is configured to process the data representing the protein graph 100 to update the pair embeddings 102, i.e., to generate updated pair embeddings 106.

Generally, a graph neural network 104 can be configured to receive data representing an input graph (e.g., a protein graph), to update the embeddings associated with the edges of the graph at each of one or more time steps, and to output the updated embeddings associated with the edges, i.e., as of the final time step. For convenience, the graph neural network may be referred to as receiving and processing an input graph, rather than receiving and processing data representing an input graph.

The graph neural network 104 updates the edge embeddings at each time step by neural network operations that are parametrized by a set of graph neural network parameters and that depend on both: (i) the topology of the graph, and (ii) the current embeddings associated with the edges in the graph. The topology of the graph refers to the arrangement of nodes and edges in the graph.

To update the current embedding associated with an edge in the graph at a time step, the graph neural network 104 can generate a respective representation of each of multiple cycles in the graph that include the edge. A “cycle” in a graph refers to a sequence of edges in a graph such that: (i) the target node of each edge in the sequence is the source node of the next edge in the sequence, and (ii) the target node of the last edge in the sequence is the source node of the first edge in the sequence, i.e., such that sequence of edges form a loop in the graph. The graph neural network 104 then updates the embedding for the edge using the representations of the cycles in the graph that include the edge.

In some implementations, the graph neural network 104 is configured to generate a respective representation of every cycle in the graph that includes the edge and that has a predefined length. The “length” of a cycle refers to the number of edges in the cycle. The predefined length can be, e.g., 3, 4, 5, or any other appropriate length.

In some implementations, the graph neural network 104 is configured to generate a respective representation of every cycle in the graph that includes the edge and that at most a predefined length. The predefined length can be, e.g., 3, 4, 5, or any other appropriate length.

The graph neural network 104 can generate a representation of a cycle in the graph by processing the embeddings associated with some or all of the edges in the cycle in accordance with values of a set of graph neural network parameters. For example, to update the embedding h_{i,j} associated with the edge from node i to node j, the graph neural network 104 can generate a representation for the cycle (h_{i,j},h_{j,k},h_{k,i}) (i.e., where h_{j,k} is the embedding associated with the edge from node j to node k and h_{k,i} is the embedding associated with the edge from node k to node i) as:

f_{θ}_{₁}(h_{j,k})⋅f_{θ}_{₂}(h_{k,i}) (1)

where f_{θ}_{₁}(⋅) is an operation implemented by one or more neural network layers (e.g., fully connected layers) having parameter values θ₁, f_{θ}_{₂}(⋅) is an operation implemented by one or more neural network layers (e.g., fully connected layers) having parameter values θ₂, and ⋅ denotes a dot product operation.

The graph neural network 104 can update the embedding for an edge using the representations of the cycles in the graph that include the edge in any appropriate way. For example, the graph neural network 104 can update the edge embedding h by:

$$h ← h + {g}_{θ} ( h , {\sum }_{c = 1}^{C} {e}_{c} ) \qquad ( 2 )$$

where g_{θ}(⋅) is an operation implemented by one or more neural network layers (e.g., fully connected layers) having parameter values θ, {c}_{c=1}^{C} indexes the cycles that include the edge, and e_{c} is the representation of cycle c. (The data being added to the edge embedding h, i.e., on the right-hand side of equation (2), can be referred to as a “residual embedding”).

Processing the protein graph 100 using the graph neural network 104 to generate updated pair embeddings 106 enriches the information content of the pair embeddings associated with the edges of the protein graph 100 by sharing information between the pair embeddings.

For example, as described above, the pair embedding corresponding to a pair of amino acids characterizes the relationship between the pair of amino acids, and in particular, the distance between the pair of amino acids in the protein structure. The spatial distances between pairs of amino acids in the protein structure have a “transitive” relationship, e.g., because amino acid i being close to amino acid j in the protein structure, and amino acid j being close to amino acid k in the protein structure, can imply that amino acid i is close to amino acid k in the protein structure. Updating the pair embeddings along cycles in the protein graph enables the graph neural network 104 to exploit this transitive relationship to effectively enrich the information content of the pair embeddings. For example, the graph neural network 104 can exploit the transitive relationship between amino acids i, j, and k described above by updating the corresponding pair embeddings along the cycle in the protein graph formed by the edges that connect AA node i to AA node j, AA node j to AA node k, and AA node k to AA node i.

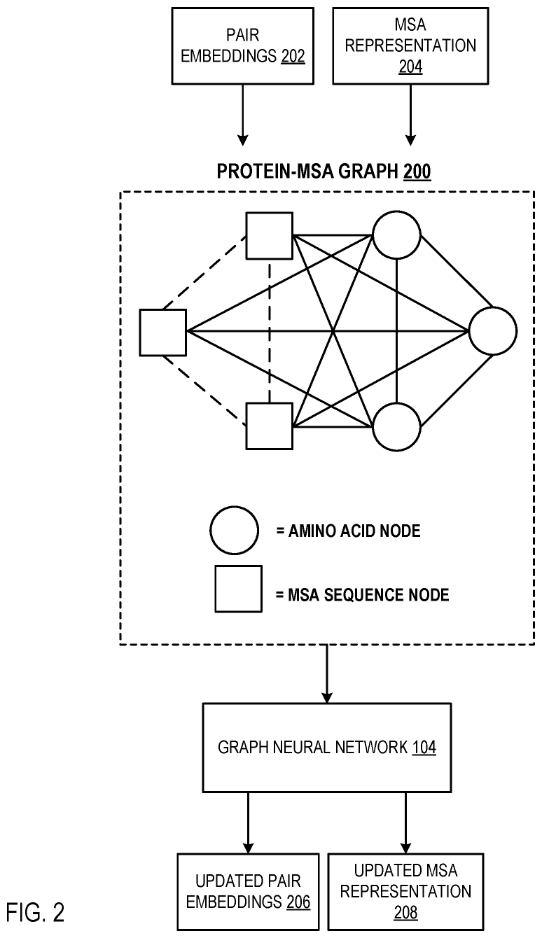

FIG. 2 shows a “protein-MSA graph” 200 that jointly represents a protein and MSAs corresponding to the amino acid chains of the protein. The protein-MSA graph 200 includes respective nodes corresponding to: (i) each amino acid in the protein, and (ii) the respective MSA sequence corresponding to each row of the MSA representation for the protein.

For convenience, nodes in the protein-MSA graph 200 corresponding to amino acids in the protein may be referred to as “amino acid (AA) nodes” or “AA nodes,” and nodes in the protein-MSA graph 200 corresponding to MSA sequences may be referred to as “MSA sequence nodes.”

The set of AA nodes in the protein-MSA graph 200 can be fully connected, i.e., such that the protein-MSA graph 200 includes a respective edge between each pair of AA nodes. Each MSA sequence node can be connected by an edge to each AA node. Optionally, each pair of MSA sequence nodes can be connected by an edge.

As with the protein graph described with reference to FIG. 1, the set of pair embeddings 202 for the protein can be associated with edges in the protein-MSA graph, i.e., such that pair embedding 202 for amino acid pair (i,j) is associated with the edge pointing from the AA node representing amino acid i to the AA node representing amino acid j.

The embeddings of the MSA representation 204 for the protein can also be associated with edges in the protein-MSA graph, i.e., such that the embedding in the MSA representation 204 corresponding to MSA sequence—amino acid pair (i,j) is associated with the edge between MSA sequence node i and AA node j.

If pairs of MSA sequence nodes in the protein-MSA graph 200 are connected by edges, then to initialize embeddings corresponding to edges between pairs of MSA sequence nodes, the system can process the MSA sequences associated with the protein to construct a phylogenetic tree, i.e., such that each node in the tree is associated with a respective MSA sequence. The system can initialize the embedding for an edge that connects a first MSA sequence node representing a first MSA sequence to a second MSA sequence node representing a second MSA sequence by determining the distance between the first MSA sequence and the second MSA sequence in the phylogenetic tree. The system can then initialize the embedding for the edge, e.g., as a one-hot embedding representing the distance between the first MSA sequence and the second MSA sequence in the phylogenetic tree. The distance between two nodes in a phylogenetic tree can refer to the minimum number of nodes in the tree that can be traversed to move from the first node to the second node. The embeddings associated with edges connecting MSA sequence nodes in the protein-MSA graph may be referred to as phylogenetic embeddings.

The system can provide data representing the protein-MSA graph 200 (i.e., including the pair embeddings and the embeddings of the MSA representation) to a graph neural network 104 that processes the protein-MSA graph 200 to generate updated pair embeddings 206 and an updated MSA representation 208. The updated MSA representation 208 is composed of the updated embeddings associated with the edges connecting MSA sequence nodes and AA nodes in the protein-MSA graph 200. If pairs of MSA sequence nodes are connected in the protein-MSA graph 200 and are associated with phylogenetic embeddings, then the graph neural network 104 can further output updated phylogenetic embeddings. The operations that can be performed by the graph neural network 104 to update the embeddings associated with the edges in the protein-MSA graph 200 are described in more detail with reference to FIG. 1.

In some implementations, the graph neural network 104 processes the protein-MSA graph 200 to update the embeddings associated with all the edges in the protein-MSA graph 200, i.e., thereby generating updated pair embeddings 206 and an updated MSA representation 208.

In some implementations, the graph neural network 104 processes the protein-MSA graph 200 to update only the embeddings associated with the edges connecting pairs of AA nodes, i.e., without updating the embeddings associated with the edges connecting MSA sequence nodes to AA nodes or the embeddings associated with edges connecting pairs of MSA sequence nodes. In these implementations, the graph neural network 104 outputs updated pair embeddings 206, but leaves the MSA representation unchanged.

In some implementations, the graph neural network 104 processes the protein-MSA graph 200 to update only the embeddings associated with the edges connecting MSA sequence nodes to AA nodes (and, optionally, embeddings associated with edges connecting pairs of MSA sequence nodes), i.e., without updating the embeddings associated with edges connecting pairs of AA nodes. In these implementations, the graph neural network 104 outputs an updated MSA representation 208, but leaves the pair embeddings 202 unchanged.

Processing the protein-MSA graph 200 using the graph neural network 104 to generate updated pair embeddings 206 and an updated MSA representation 208 can enrich the information content of the pair embeddings and the MSA representation by sharing information between them.

FIG. 3 shows an example protein structure prediction system 300 that, as part of predicting the structure of a protein, uses graph neural networks to process protein graphs and/or protein-MSA graphs for the protein. The protein structure prediction system 300 is an example of a system implemented as computer programs on one or more computers in one or more locations in which the systems, components, and techniques described below are implemented.

The system 300 is configured to process data defining one or more amino acid chains 304 of a protein 302 to generate a set of structure parameters 314 that define a predicted protein structure 316, i.e., a prediction of the structure of the protein 302. That is, the predicted structure 316 of the protein 302 can be defined by a set of structure parameters 314 that collectively define a predicted three-dimensional structure of the protein after the protein undergoes protein folding.

The structure parameters 314 defining the predicted protein structure 316 may include, e.g., location parameters and rotation parameters for each amino acid in the protein 302, a distance map that characterizes estimated distances between each pair of amino acids in the protein, a respective spatial location of each atom in each amino acid in the structure of the protein, or a combination thereof, as described above.

To generate the structure parameters 314 defining the predicted protein structure 316, the system 300 generates: (i) a multiple sequence alignment (MSA) representation 306 for the protein (in some implementations), and (ii) a set of “pair” embeddings 308 for the protein.

The MSA representation 306 for the protein includes a respective representation of a MSA for each amino acid chain in the protein. A MSA representation for an amino acid chain in the protein can be represented as a M×N array of embeddings (i.e., a 2-D array of embeddings having M rows and N columns), where N is the number of amino acids in the amino acid chain. Each row of the MSA representation can correspond to a respective MSA sequence for the amino acid chain in the protein. The system can initialize a MSA representation for an amino acid chain in any appropriate way. For example, the system can initialize the embedding at each position (i,j) in the MSA representation of the amino acid chain to be a value e.g. a one-hot vector defining the identity of the amino acid at position j in MSA sequence i.

The system 300 generates the MSA representation 306 for the protein 302 from the MSA representations for the amino acid chains in the protein.

If the protein includes only a single amino acid chain, then the system 300 can identify the MSA representation 306 for the protein 302 as being the MSA representation for the single amino acid chain in the protein.

If the protein includes multiple amino acid chains, then the system 300 can generate the MSA representation 306 for the protein by assembling the MSA representations for the amino acid chains in the protein into a block diagonal 2-D array of embeddings, i.e., where the MSA representations for the amino acid chains in the protein form the blocks on the diagonal. The system 300 can initialize the embeddings at each position in the 2-D array outside the blocks on the diagonal to be a default embedding, e.g., a vector of zeros. The amino acid chains in the protein can be assigned an arbitrary ordering, and the MSA representations of the amino acid chains in the protein can be ordered accordingly in the block diagonal matrix. For example, the MSA representation for the first amino acid chain (i.e., according to the ordering) can be the first block on the diagonal, the MSA representation for the second amino acid chain can be the second block on the diagonal, and so on.

Generally, the MSA representation 306 for the protein can be represented as a 2-D array of embeddings. Throughout this specification, a “row” of the MSA representation for the protein refers to a row of a 2-D array of embeddings defining the MSA representation for the protein. Similarly, a “column” of the MSA representation for the protein refers to a column of a 2-D array of embeddings defining the MSA representation for the protein.

The set of pair embeddings 308 includes a respective pair embedding corresponding to each pair of amino acids in the protein 302. The pair embedding for each edge may be initialized, and optionally updated, based at least in part on an MSA representation for the protein that represents a respective MSA corresponding to each amino acid chain in the protein. For example the system can initialize the pair embeddings, e.g., by applying an outer product mean operation to the MSA representation 306, and identifying the pair embeddings 308 as the result of the outer product mean operation. To compute the outer product mean, the system generates a tensor A(⋅), e.g., given by:

$$A ( res 1 , res 2 , ch 1 , ch 2 ) = \frac{1}{\vert rows \vert} {\sum }_{rows} LeftAct ( row , res 1 , ch 1 ) \cdot RightAct ( row , res 2 , ch 2 )$$

where res1, res2∈{1, . . . , N}, where N is the number of amino acids in the protein, ch1, ch2∈{1, . . . , C}, where C is the number of channels in each embedding of the MSA representation, |rows| is the number rows in the MSA representation, LeftAct(row,res1,ch1) is a linear operation (e.g., defined by a matrix multiplication) applied to the channel ch1 of the embedding of the MSA representation located at the row indexed by “row” and the column indexed by “res1”, and RightAct(row,res2,ch2) is a linear operation (e.g., defined by a matrix multiplication) applied to the channel ch2 of the embedding of the MSA representation located at the row indexed by “row” and the column indexed by “res2”. The result of the outer product mean is generated by flattening and linearly projecting the (ch1,ch2) dimensions of the tensor A. Optionally, the system can perform one or more Layer Normalization operations (e.g., as described with reference to Jimmy Lei Ba et al., “Layer Normalization,” arXiv:1607.06450) as part of computing the outer product mean.

The system 300 can determine the predicted structure of the protein based on the pair embeddings. However in implementations the system 300 generates the structure parameters 314 defining the predicted protein structure 316 using both the MSA representation 306 and the pair embeddings 308, because both have complementary properties. The structure of the MSA representation 306 can explicitly depend on the number of amino acid chains in the MSAs corresponding to each amino acid chain in the protein. Therefore, the MSA representation 306 may be inappropriate for use in directly predicting the protein structure, because the protein structure 316 has no explicit dependence on the number of amino acids chains in the MSAs. In contrast, the pair embeddings 308 characterize relationships between respective pairs of amino acids in the protein 302 and are expressed without explicit reference to the MSAs, and are therefore a convenient and effective data representation for use in predicting the protein structure 316.

The system 300 processes the MSA representation 306 and the pair embeddings 308 using an embedding neural network 400, in accordance with the values of a set of parameters of the embedding neural network 400, to update the MSA representation 306 and the pair embeddings 308. That is, the embedding neural network 400 processes the MSA representation 306 and the pair embeddings 308 to generate an updated MSA representation 310 and updated pair embeddings 312.

The embedding neural network 400 updates the MSA representation 306 and the pair embeddings 308 by sharing information between the MSA representation 306 and the pair embeddings 308. For example, the embedding neural network 400 can alternate between updating the current MSA representation 306 based on the current pair embeddings 308, and updating the current pair embeddings 308 based on the current MSA representation 306.

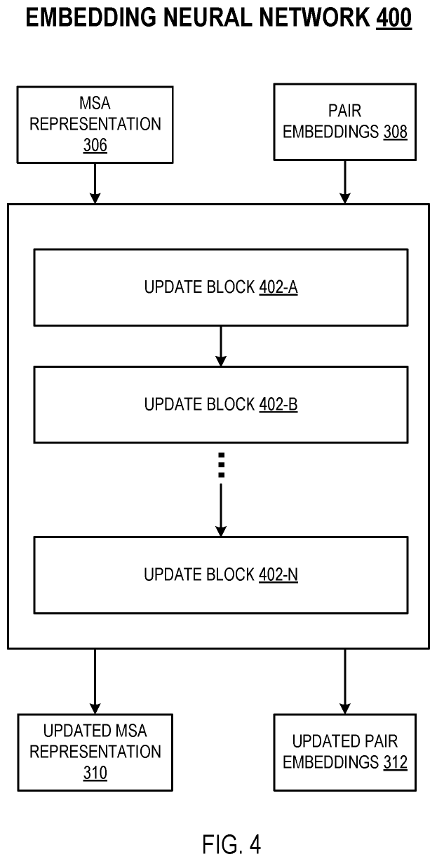

An example architecture of the embedding neural network 400 is described in more detail with reference to FIG. 4. As will be described with reference to FIG. 4, the embedding neural network 400 can update the pair embeddings, the MSA representation, or both, by processing a protein graph and/or a protein-MSA graph for the protein using graph neural networks.

The system 300 generates a network input for a folding neural network 800 from the updated pair embeddings 312, the updated MSA representation 310, or both, and processes the network input using the folding neural network 800 to generate the structure parameters 314 defining the predicted protein structure.

In some implementations, the folding neural network 800 processes the updated pair embeddings 312 to generate a distance map that includes, for each pair of amino acids in the protein, a probability distribution over a set of possible distances between the pair of amino acids in the protein structure. For example, to generate the probability distribution over the set of possible distances between a pair of amino acids in the protein structure, the folding neural network may apply one or more fully-connected neural network layers to an updated pair embedding 312 corresponding to the pair of amino acids.

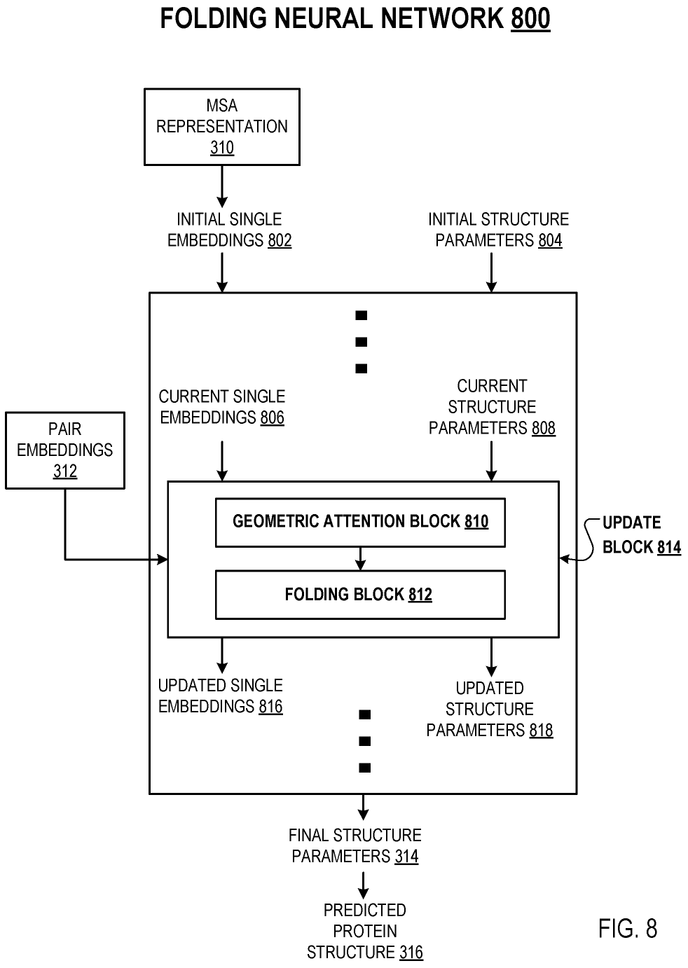

In some implementations, the folding neural network 800 generates the structure parameters 314 by processing a network input derived from both the updated MSA representation 310 and the updated pair embeddings 312 using a geometric attention operation that explicitly reasons about the 3-D geometry of the amino acids in the protein structure. An example architecture of the folding neural network 800 that implements a geometric attention mechanism is described with reference to FIG. 8.

A training engine may train the protein structure prediction system 300 from end-to-end to optimize an objective function referred to herein as a structure loss. The training engine may train the system 300 on a set of training data including multiple training examples. Each training example may specify: (i) a training input that includes an initial MSA representation and initial pair embeddings for a protein, and (ii) a target protein structure that should be generated by the system 300 by processing the training input. Target protein structures used for training the system 300 may be determined using experimental techniques, e.g., x-ray crystallography or cryo-EM.

The structure loss may characterize a similarity between: (i) a predicted protein structure generated by the system 300, and (ii) the target protein structure that should have been generated by the system.

For example, if the predicted structure parameters define predicted location parameters and predicted rotation parameters for each amino acid in the protein, then the structure loss [symbol] _{structure} may be given by:

$${ℒ}_{structure} = \frac{1}{{N}^{2}} {\sum }_{i , j = 1}^{N} {( 1 - \frac{\vert {t}_{i j} - \vert}{A} )}_{+} \qquad ( 3 )$$

$${t}_{ij} = {R}_{i}^{- 1} ( {t}_{j} - {t}_{i} ) \qquad ( 4 )$$

$$= {\tilde{R}}_{ι}^{- 1} ( {\tilde{t}}_{j} - \tilde{{t}_{ι}} ) \qquad ( 5 )$$

where N is the number of amino acids in the protein, tᵢ denote the predicted location parameters for amino acid i, Rᵢ denotes a 3×3 rotation matrix specified by the predicted rotation parameters for amino acid i, [symbol] are the target location parameters for amino acid i, \tilde{R}ₗ denotes a 3×3 rotation matrix specified by the target rotation parameters for amino acid i, A is a constant, Rᵢ⁻¹ refers to the inverse of the 3×3 rotation matrix specified by predicted rotation parameters Rᵢ, \tilde{R}ₗ⁻¹ refers to the inverse of the 3×3 rotation matrix specified by the target rotation parameters \tilde{R}ₗ, and (⋅)₊ denotes a rectified linear unit (ReLU) operation.

The structure loss defined with reference to equations (3)-(5) may be understood as averaging the loss |tᵢⱼ− [symbol] | over each pair of amino acids in the protein. The term tᵢⱼ defines the predicted spatial location of amino acid j in the predicted frame of reference of amino acid i, and [symbol] defines the actual spatial location of amino acid j in the actual frame of reference of amino acid i. These terms are sensitive to the predicted and actual rotations of amino acid i and j, and therefore carry richer information than loss terms that are only sensitive to the predicted and actual distances between amino acids.

As another example, if the predicted structure parameters define predicted spatial locations of each atom in each amino acid of the protein, then the structure loss may be an average error (e.g., squared-error) between: (i) the predicted spatial locations of the atoms, and (ii) the target (e.g., ground truth) spatial locations of the atoms.

Optimizing the structure loss encourages the system 300 to generate predicted protein structures that accurately approximate true protein structures.

In addition to optimizing the structure loss, the training engine may train the system 300 to optimize one or more auxiliary losses. The auxiliary losses may penalize predicted structures having characteristics that are unlikely to occur in the natural world, e.g., based on the bond angles and/or bond lengths of the bonds between the atoms in the amino acids in the predicted structures, or based on the proximity of the atoms in different amino acids in the predicted structures.

The training engine may train the structure prediction system 300 on the training data over multiple training iterations, e.g., using stochastic gradient descent training techniques.

FIG. 4 shows an example architecture of an embedding neural network 400 that is configured to process the MSA representation 306 and the pair embeddings 308 to generate the updated MSA representation 310 and the updated pair embeddings 312.

The embedding neural network 400 includes a sequence of update blocks 402-A-N. Throughout this specification, a “block” refers to a portion of a neural network, e.g., a subnetwork of the neural network that includes one or more neural network layers.

Each update block in the embedding neural network is configured to receive a block input that includes a MSA representation and a pair embedding, and to process the block input to generate a block output that includes an updated MSA representation and an updated pair embedding.

The embedding neural network 400 provides the MSA representation 306 and the pair embeddings 308 included in the network input of the embedding neural network 400 to the first update block (i.e., in the sequence of update blocks). The first update block processes the MSA representation 306 and the pair embeddings 308 to generate an updated MSA representation and updated pair embeddings.

For each update block after the first update block, the embedding neural network 400 provides the update block with the MSA representation and the pair embeddings generated by the preceding update block, and provides the updated MSA representation and the updated pair embeddings generated by the update block to the next update block.

The embedding neural network 400 gradually enriches the information content of the MSA representation 306 and the pair embeddings 308 by repeatedly updating them using the sequence of update blocks 402-A-N.

The embedding neural network 400 may provide the updated MSA representation 310 and the updated pair embeddings 312 generated by the final update block (i.e., in the sequence of update blocks) as the network output.

Generally, the update blocks in the embedding neural network are not all required to have the same architecture, and in particular, different update blocks can have different architectures. Some or all of the update blocks use a graph neural network to process a protein graph and/or a protein MSA graph as part of updating the current MSA representation and current pair embeddings.

In some implementations, one or more of the update blocks of the embedding neural network process a protein-MSA graph, where the edges in the protein-MSA graph are associated with embeddings defined by the current pair embeddings and the current MSA representation, to generate updated pair embeddings and an updated MSA representation.

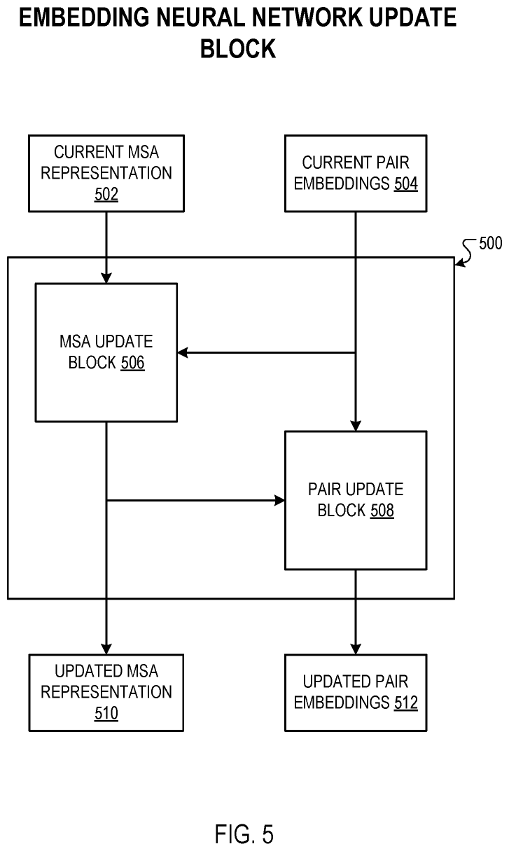

FIG. 5 shows another possible architecture of an update block 500 of the embedding neural network 400.

The update block 500 receives a block input that includes the current MSA representation 502 and the current pair embeddings 504, and processes the block input to generate the updated MSA representation 510 and the updated pair embeddings 512.

The update block 500 includes an MSA update block 506 and a pair update block 508.

The MSA update block 506 updates the current MSA representation 502 using the current pair embeddings 504, and the pair update block 508 updates the current pair embeddings 504 using the updated MSA representation 510 (i.e., that is generated by the MSA update block 506).

Generally, the MSA representation and the pair embeddings can encode complementary information. For example, the MSA representation can encode information about the correlations between the identities of the amino acids in different positions among a set of evolutionarily-related amino acid chains, and the pair embeddings can encode information about the inter-relationships between the amino acids in the protein. The MSA update block 506 enriches the information content of the MSA representation using complementary information encoded in the pair embeddings, and the pair update block 508 enriches the information content of the pair embeddings using complementary information encoded in the MSA representation. As a result of this enrichment, the updated MSA representation and the updated pair embedding encode information that is more relevant to predicting the protein structure.

The update block 500 is described herein as first updating the current MSA representation 502 using the current pair embeddings 504, and then updating the current pair embeddings 504 using the updated MSA representation 510. The description should not be understood as limiting the update block to performing operations in this sequence, e.g., the update block could first update the current pair embeddings using the current MSA representation, and then update the current MSA representation using the updated pair embeddings.

The update block 500 is described herein as including an MSA update block 506 (i.e., that updates the current MSA representation) and a pair update block 508 (i.e., that updates the current pair embeddings). The description should not be understood to limiting the update block 500 to include only one MSA update block or only one pair update block. For example, the update block 500 can include multiple MSA update blocks that update the MSA representation multiple times before the MSA representation is provided to a pair update block for use in updating the current pair embeddings. As another example, the update block 500 can include multiple pair update blocks that update the pair embeddings multiple times using the MSA representation.

The MSA update block 506 and the pair update block 508 can have any appropriate architectures that enable them to perform their described functions.

In some implementations, the MSA update block 506, the pair update block 508, or both, include one or more “self-attention” blocks. As used throughout this document, a self-attention block generally refers to a neural network block that updates a collection of embeddings, i.e., that receives a collection of embeddings and outputs updated embeddings. To update a given embedding, the self-attention block can determine a respective “attention weight” between the given embedding and each of one or more selected embeddings, and then update the given embedding using: (i) the attention weights, and (ii) the selected embeddings. For convenience, the self-attention block may be said to update the given embedding using attention “over” the selected embeddings.

For example, a self-attention block may receive a collection of input embeddings {xᵢ}ᵢ₌₁^{N}, where N is the number of amino acids in the protein, and to update embedding xᵢ, the self-attention block may determine attention weights [a_{i,j}]ⱼ₌₁^{N} where a_{i,j} denotes the attention weight between xᵢ and xⱼ, as:

$${[ {a}_{i , j} ]}_{j = 1}^{N} = softmax ( \frac{( {W}_{q} {x}_{i} ) {K}^{T}}{c} ) \qquad ( 6 )$$

$${K}^{T} = {[ {W}_{k} {x}_{j} ]}_{j = 1}^{N} \qquad ( 7 )$$

where W_{q} and Wₖ are learned parameter matrices, softmax(⋅) denotes a soft-max normalization operation, and c is a constant. Using the attention weights, the self-attention layer may update embedding xᵢ as:

$${x}_{i} ← {\sum }_{j = 1 … N} {a}_{i , j} \cdot ( {W}_{v} {x}_{j} ) \qquad ( 8 )$$

where Wᵥ is a learned parameter matrix. (W_{q}xᵢ can be referred to as the “query embedding” for input embedding xᵢ, Wₖxⱼ can be referred to as the “key embedding” for input embedding xᵢ, and Wᵥxⱼ can be referred to as the “value embedding” for input embedding xᵢ).

The parameter matrices W_{q} (the “query embedding matrix”), Wₖ (the “key embedding matrix”), and Wᵥ (the “value embedding matrix”) are trainable parameters of the self-attention block. The parameters of any self-attention blocks included in the MSA update block 506 and the pair update block 508 can be understood as being parameters of the update block 500 that can be trained as part of the end-to-end training of the protein structure prediction system 300 described with reference to FIG. 3. Generally, the (trained) parameters of the query, key, and value embedding matrices are different for different self-attention blocks, e.g., such that a self-attention block included in the MSA update block 506 can have different query, key, and value embedding matrices with different parameters than a self-attention block included in the pair update block 508.

In some implementations, the MSA update block 506, the pair update block 508, or both, include one or more self-attention blocks that are conditioned on the pair embeddings, i.e., that implement self-attention operations that are conditioned on the pair embeddings. To condition a self-attention operation on the pair embeddings, the self-attention block can process the pair embeddings to generate a respective “attention bias” corresponding to each attention weight. For example, in addition to determining the attention weights [a_{i,j}]ⱼ₌₁^{N} in accordance with equations (6)-(7), the self-attention block can generate a corresponding set of attention biases [b_{i,j}]ⱼ₌₁^{N}, where b_{i,j} denotes the attention bias between xᵢ and xⱼ. The self-attention block can generate the attention bias b_{i,j} by applying a learned parameter matrix to the pair embedding h_{i,j}, i.e., for the pair of amino acids in the protein indexed by (i,j).

The self-attention block can determine a set of “biased attention weights” [c_{i,j}]ⱼ₌₁^{N}, where c_{i,j} denotes the biased attention weight between xᵢ and xⱼ, e.g., by summing (or otherwise combining) the attention weights and the attention biases. For example, the self-attention block can determine the biased attention weight c_{i,j} between embeddings xᵢ and xⱼ as:

c_{i,j}=a_{i,j}+b_{i,j}

where a_{i,j} is the attention weight between xᵢ and xⱼ and b_{i,j} is the attention bias between xᵢ and xⱼ. The self-attention block can update each input embedding xᵢ using the biased attention weights, e.g.:

$${x}_{i} ← {\sum }_{j = 1 … N} {c}_{i , j} \cdot ( {W}_{v} {x}_{j} ) \qquad ( 9 )$$

where Wᵥ is a learned parameter matrix.

Generally, the pair embeddings encode information characterizing the structure of the protein and the relationships between the pairs of amino acids in the structure of the protein. Applying a self-attention operation that is conditioned on the pair embeddings to a set of input embeddings allows the input embeddings to be updated in a manner that is informed by the protein structural information encoded in the pair embeddings. The update blocks of the embedding neural network can use the self-attention blocks that are conditioned on the pair embeddings to update and enrich the MSA representation and the pair embeddings themselves.

Optionally, a self-attention block can have multiple “heads” that each generate a respective updated embedding corresponding to each input embedding, i.e., such that each input embedding is associated with multiple updated embeddings. For example, each head may generate updated embeddings in accordance with different values of the parameter matrices W_{q}, Wₖ, and Wᵥ that are described with reference to equations (6)-(9). A self-attention block with multiple heads can implement a “gating” operation to combine the updated embeddings generated by the heads for an input embedding, i.e., to generate a single updated embedding corresponding to each input embedding. For example, the self-attention block can process the input embeddings using one or more neural network layers (e.g., fully connected neural network layers) to generate a respective gating value for each head. The self-attention block can then combine the updated embeddings corresponding to an input embedding in accordance with the gating values. For example, the self-attention block can generate the updated embedding for an input embedding xᵢ as:

$${\sum }_{k = 1}^{K} {α}_{k} \cdot {x}_{i}^{next} \qquad ( 10 )$$

where k indexes the heads, aₖ is the gating value for head k, and xᵢ^{next} is the updated embedding generated by head k for input embedding xᵢ.

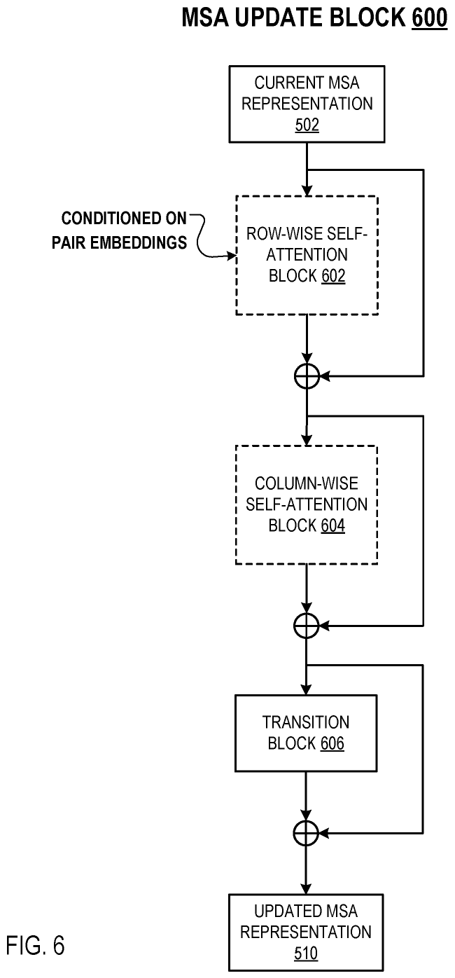

An example architecture of a MSA update block 506 that uses self-attention blocks conditioned on the pair embeddings is described with reference to FIG. 6. The example MSA update block described with reference to FIG. 6 updates the current MSA representation based on the current pair embeddings by processing the rows of the current MSA representation using a self-attention block that is conditioned on the current pair embeddings.

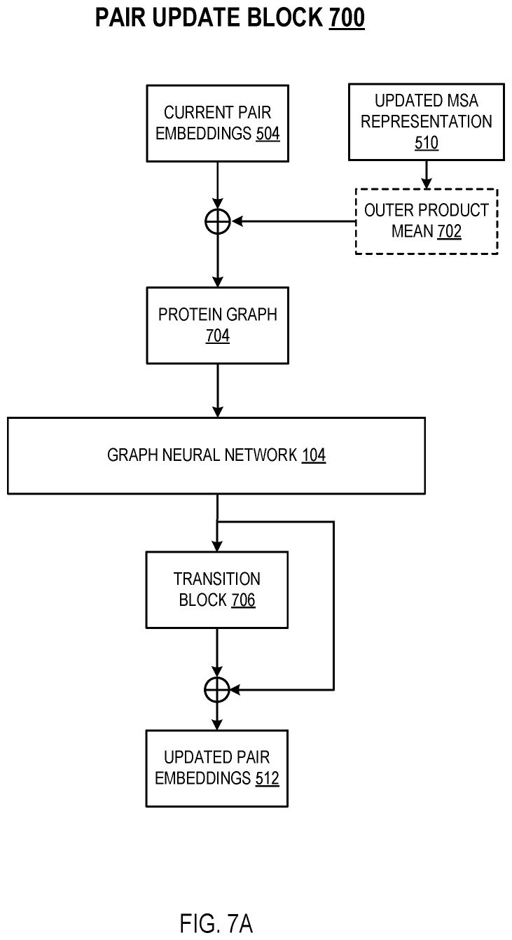

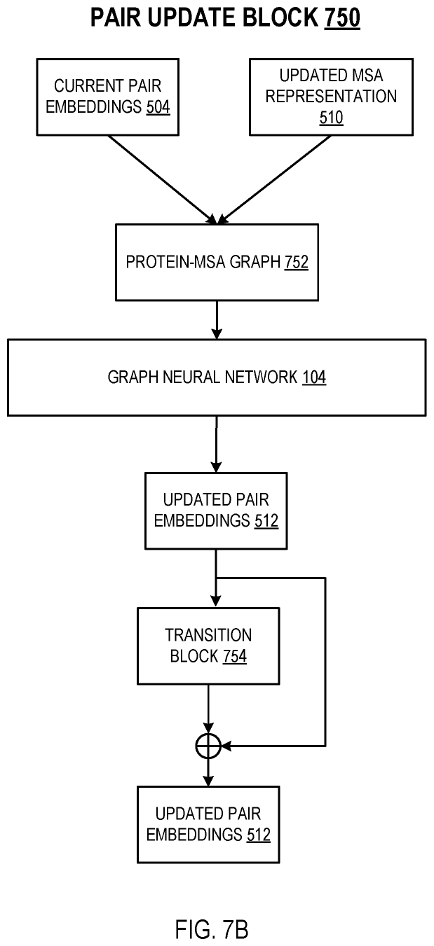

Example architectures of the pair update block 500 are described with reference to FIG. 7A and FIG. 7B. FIG. 7A shows an example architecture of a pair update block that updates the current pair embeddings by processing a protein graph for the protein using a graph neural network. FIG. 7B shows an example architecture of a pair update block that updates the current pair embeddings by processing a protein-MSA graph for the protein using a graph neural network.

FIG. 6 shows an example architecture of a MSA update block 506. The MSA update block 506 is configured to receive the current MSA representation 502, to update the current MSA representation 502 based (at least in part) on the current pair embedding.

To update the current MSA representation 502, the MSA update block 506 updates the embeddings in each row of the current MSA representation using a self-attention operation (i.e., a “row-wise” self-attention operation) that is conditioned on the current pair embeddings. More specifically, the MSA update block 506 provides the embeddings in each row of the current MSA representation 502 to a “row-wise” self-attention block 602 that is conditioned on the current pair embeddings, e.g., as described with reference to FIG. 5, to generate updated embeddings for each row of the current MSA representation 502. Optionally, the MSA update block can add the input to the row-wise self-attention block 602 to the output of the row-wise self-attention block 602. Conditioning the row-wise self-attention block 602 on the current pair embeddings enables the MSA update block 506 to enrich the current MSA representation 502 using information from the current pair embeddings.

The MSA update block then updates the embeddings in each column of the current MSA representation using a self-attention operation (i.e., a “column-wise” self-attention operation) that is not conditioned on the current pair embeddings. More specifically, the MSA update block 506 provides the embeddings in each column of the current MSA representation 502 to a “column-wise” self-attention block 604 that is not conditioned on the current pair embeddings to generate updated embeddings for each column of the current MSA representation 502. As a result of not being conditioned on the current pair embeddings, the column-wise self-attention block 604 generates updated embeddings for each column of the current MSA representation using attention weights (e.g., as described with reference to equations (6)-(8)) rather than biased attention weights (e.g., as described with reference to equation (9)). Optionally, the MSA update block can add the input to the column-wise self-attention block 604 to the output of the column-wise self-attention block 604.

The MSA update block then processes the current MSA representation 502 using a transition block, e.g., that applies one or more fully-connected neural network layers to the current MSA representation 502. Optionally, the MSA update block 506 can add the input to the transition block 606 to the output of the transition block 606.

The MSA update block can output the updated MSA representation 510 resulting from the operations performed by the row-wise self-attention block 602, the column-wise self-attention block 604, and the transition block 606.

FIG. 7A shows an example architecture of a pair update block 700 that updates the current pair embeddings by processing a protein graph for the protein using a graph neural network. The pair update block 700 is configured to receive the current pair embeddings 504, and to update the current pair embeddings 504 based (at least in part) on the updated MSA representation 512.

To update the current pair embeddings 504, the pair update block 700 applies an outer product mean operation 702 to the updated MSA representation 512 and adds the result of the outer product mean operation 702 to the current pair embeddings 504.

The outer product mean operation defines a sequence of operations that, when applied to an MSA representation represented as an L×N array of embeddings, generates an N×N array of embeddings, i.e, where N is the number of amino acids in the protein. The current pair embeddings 504 can also be represented as an N×N array of embeddings, and adding the result of the outer product mean 702 to the current pair embeddings 504 refers to summing the two N×N arrays of embeddings.

To compute the outer product mean, the pair update block generates a tensor A(⋅), e.g., given by:

$$A ( res 1 , res 2 , ch 1 , ch 2 ) = \frac{1}{\vert rows \vert} {\sum }_{rows} LeftAct ( row , res 1 , ch 1 ) \times RightAct ( row , res 2 , ch 2 )$$

where res1, res2∈{1, . . . , N}, ch1, ch2∈{1, . . . , C}, where C is the number of channels in each embedding of the MSA representation, |rows| is the number rows in the MSA representation, LeftAct(row,res1,ch1) is a linear operation (e.g., defined by a matrix multiplication) applied to the channel ch1 of the embedding of the MSA representation located at the row indexed by “row” and the column indexed by “res1”, and RightAct(row,res2,ch2) is a linear operation (e.g., defined by a matrix multiplication) applied to the channel ch2 of the embedding of the MSA representation located at the row indexed by “row” and the column indexed by “rest”, and z× is an outer product operation. The result of the outer product mean is generated by flattening and linearly projecting the (ch1, ch2) dimensions of the tensor A. Optionally, the pair update block can perform one or more Layer Normalization operations (e.g., as described with reference to Jimmy Lei Ba et al., “Layer Normalization,” arXiv:1607.06450) as part of computing the outer product mean.

Generally, the updated MSA representation 512 encodes information about the correlations between the identities of the amino acids in different positions among a set of evolutionarily-related amino acid chains. The information encoded in the updated MSA representation 512 is relevant to predicting the structure of the protein, and by incorporating the information encoded in the updated MSA representation into the current pair embeddings (i.e., by way of the outer product mean 702), the pair update block 700 can enhance the information content of the current pair embeddings.

After updating the current pair embeddings 504 using the updated MSA representation (i.e., by way of the outer product mean 702), the pair update block 700 generates a protein graph 704, i.e., as described with reference to FIG. 1, where the edges in the protein graph 704 are associated with the current pair embeddings. The pair update block then processes the protein graph 704 using a graph neural network 104 to update the current pair embeddings.

The pair update block 700 then processes the current pair embeddings using a transition block, e.g., that applies one or more fully-connected neural network layers to the current pair embeddings. Optionally, the pair update block 700 can add the input to the transition block 706 to the output of the transition block 706.

The pair update block can output the updated pair embeddings 512 resulting from the operations performed by the graph neural network 104 and the transition block 706.

FIG. 7B shows an example architecture of a pair update block that updates the current pair embeddings by processing a protein-MSA graph for the protein using a graph neural network. The pair update block 750 is configured to receive the current pair embeddings 504, and to update the current pair embeddings 504 based (at least in part) on the updated MSA representation 510.

The pair update block 750 uses the current pair embeddings 504 and the updated MSA representation to generate a protein-MSA graph 752 for the protein. As described with reference to FIG. 2, the pair update block 750 can associate the current pair embeddings 504 with edges connecting corresponding pairs of AA nodes in the protein-MSA graph 752, and the pair update block 750 can associate the embeddings of the updated MSA representation 510 with edges connecting corresponding MSA sequence node—AA node pairs in the protein-MSA graph 752.

The pair update block 750 processes the protein-MSA graph 752 using a graph neural network 104 to update the current pair embeddings 504, i.e., to generate updated pair embeddings 512. The operations performed by the graph neural network 104 cause the current pair embeddings to be updated based at least in part on the MSA representation, e.g., because the current pair embeddings are updated based on cycles in the graph that include edges that are associated with embeddings of the MSA representation 510.

Optionally, but not necessarily, the graph neural network 104 can update the embeddings associated with edges connecting MSA sequence nodes to AA nodes to update the MSA representation.

The pair update block 750 then processes the pair embeddings 504 using a transition block, e.g., that applies one or more fully-connected neural network layers to the pair embeddings. Optionally, the pair update block 750 can add the input to the transition block 754 to the output of the transition block 754.

The pair update block can output the updated pair embeddings 512 resulting from the operations performed by the graph neural network 104 and the transition block 754.

FIG. 8 shows an example architecture of a folding neural network 800 that generates a set of structure parameters 314 that define the predicted protein structure 316. For example in implementations the folding neural network 800 determines the predicted structure of the protein based on the pair embeddings by processing an input comprising a respective pair embedding 312 for each pair of amino acids in the protein to generate values of the structure parameters 314. The folding neural network 800 can be included in the protein structure prediction system described with reference to FIG. 3.

In implementations the folding neural network 800 generates structure parameters that can include: (i) location parameters, and (ii) rotation parameters, for each amino acid in the protein. As described earlier, the location parameters for an amino acid may specify a predicted 3-D spatial location of a specified atom in the amino acid in the structure of the protein. The rotation parameters for an amino acid may specify the predicted “orientation” of the amino acid in the structure of the protein. More specifically, the rotation parameters may specify a 3-D spatial rotation operation that, if applied to the coordinate system of the location parameters, causes the three “main chain” atoms in the amino acid to assume fixed positions relative to the rotated coordinate system.

The folding neural network 800 receives an input that includes: (i) a respective pair embedding 312 for each pair of amino acids in the protein, (ii) initial values of a “single” embedding 802 for each amino acid in the protein, and (iii) initial values of structure parameters 804 for each amino acid in the protein. The folding neural network 800 processes the input to generate final values of the structure parameters 314 that collectively characterize the predicted structure 316 of the protein.

The protein structure prediction system can provide the folding neural network 800 with the pair embeddings generated as an output of an embedding neural network, as described with reference to FIG. 4.

The protein structure prediction system can generate the initial single embeddings 802 for the amino acids from the MSA representation 310, i.e., that is generated as an output of an embedding neural network, as described with reference to FIG. 4. For example, as described above, the MSA representation 310 can be represented as a 2-D array of embeddings having a number of columns equal to the number of amino acids in the protein, where each column is associated with a respective amino acid in the protein. The protein structure prediction system can generate the initial single embedding for each amino acid in the protein by summing (or otherwise combining) the embeddings from the column of the MSA representation 310 that is associated with the amino acid. As another example, the protein structure prediction system can generate the initial single embeddings for the amino acids in the protein by extracting the embeddings from a row of the MSA representation 310 that corresponds to the amino acid sequence of the protein whose structure is being estimated.

The protein structure prediction system may generate the initial structure parameters 804 with default values, e.g., where the location parameters for each amino acid are initialized to the origin (e.g., [0,0,0] in a Cartesian coordinate system), and the rotation parameters for each amino acid are initialized to a 3×3 identity matrix.

The folding neural network 800 can generate the final structure parameters 314 by repeatedly updating the current values of the single embeddings 806 and the structure parameters 808, i.e., starting from their initial values. More specifically, the folding neural network 800 includes a sequence of update neural network blocks 814, where each update block 814 is configured to update the current single embeddings 806 (i.e., to generate updated single embeddings 816) and to update the current structure parameters 808 (i.e., to generate updated structure parameters 818). The folding neural network 800 may include other neural network layers or blocks in addition to the update blocks, e.g., that may be interleaved with the update blocks.

Each update block 814 can include: (i) a geometric attention block 810, and (ii) a folding block 812, each of which will be described in more detail next.

The geometric attention block 810 updates the current single embeddings using a “geometric” self-attention operation that explicitly reasons about the 3-D geometry of the amino acids in the structure of the protein, i.e., as defined by the structure parameters. More specifically, to update a given single embedding, the geometric attention block 810 determines a respective attention weight between the given single embedding and each of one or more selected single embeddings, where the attention weights depend on both the current single embeddings, the current structure parameters, and the pair embeddings. The geometric attention block 810 then updates the given single embedding using: (i) the attention weights, (ii) the selected single embeddings, and (iii) the current structure parameters.

To determine the attention weights, the geometric attention block 810 processes each current single embedding to generate a corresponding “symbolic query” embedding, “symbolic key” embedding, and “symbolic value” embedding. For example, the geometric attention block 810 may generate the symbolic query embedding qᵢ, symbolic key embedding kᵢ, and symbolic value embedding vᵢ for the single embedding hᵢ corresponding to the i-th amino acid as:

qᵢ=Linear(hᵢ) (11)

kᵢ=Linear(hᵢ) (12)

vᵢ=Linear(hᵢ) (13)

where Linear(⋅) refers to linear layers having independent learned parameter values.

The geometric attention block 810 additionally processes each current single embedding to generate a corresponding “geometric query” embedding, “geometric key” embedding, and “geometric value” embedding. The geometric query, geometric key, and geometric value embeddings for each single embedding are each 3-D points that are initially generated in the local reference frame of the corresponding amino acid, and then rotated and translated to a global reference frame using the structure parameters for the amino acid. For example, the geometric attention block 810 may generate the geometry query embedding qᵢ^{p}, geometric key embedding kᵢ^{p}, and geometric value embedding vᵢ^{p} for the single embedding hᵢ corresponding to the i-th amino acid as:

aᵢ^{p}=Rᵢ·Linearₚ(hᵢ)+tᵢ (14)

kᵢ^{p}=Rᵢ·Linearₚ(hᵢ)+tᵢ (15)

vᵢ^{p}=Rᵢ·Linearₚ(hᵢ)+tᵢ (16)

where Linear p(⋅) refers to linear layers having independent learned parameter values that project hᵢ to a 3-D point (the superscript p indicates that the quantity is a 3-D point), Rᵢ denotes the rotation matrix specified by the rotation parameters for the i-th amino acid, and tᵢ denotes the location parameters for the i-th amino acid.

To update the single embedding hᵢ corresponding to amino acid i, the geometric attention block 810 may generate attention weights [aⱼ]ⱼ₌₁^{N}, where N is the total number of amino acids in the protein and aⱼ is the attention weight between amino acid i and amino acid j, as:

$${[ {a}_{j} ]}_{j = 1}^{N} = softmax ( {[ \frac{{q}_{i} \cdot {k}_{j}}{\sqrt{m}} + α {\vert {q}_{i}^{p} - {k}_{j}^{p} \vert}_{2}^{2} + ( {b}_{i , j} \cdot w ) ]}_{j = 1}^{N} ) \qquad ( 17 )$$

where qᵢ denotes the symbolic query embedding for amino acid i, kⱼ denotes the symbolic key embedding for amino acid j, m denotes the dimensionality of qᵢ and kⱼ, a denotes a learned parameter, qᵢ^{p} denotes the geometric query embedding for amino acid i, kⱼ^{p} denotes the geometry key embedding for amino acid j, |⋅|₂ is an L₂ norm, and b_{i,j} is the pair embedding 116 corresponding to the pair of amino acids that includes amino acid i and amino acid j, and w is a learned weight vector (or some other learned projection operation).

Generally, the pair embedding for a pair of amino acids implicitly encodes information relating the relationship between the amino acids in the pair, e.g., the distance between the amino acids in the pair. By determining the attention weight between amino acid i and amino acid j based in part on the pair embedding for amino acids i and j, the folding neural network 800 enriches the attention weights with the information from the pair embedding and thereby improves the accuracy of the predicted folding structure.

In some implementations, the geometric attention block 810 generate multiple sets of geometric query embeddings, geometric key embeddings, and geometric value embeddings, and uses each generated set of geometric embeddings in determining the attention weights.

After generating the attention weights for the single embedding hᵢ corresponding to amino acid i, the geometric attention block 810 uses the attention weights to update the single embedding hᵢ. In particular, the geometric attention block 810 uses the attention weights to generate a “symbolic return” embedding and a “geometric return” embedding, and then updates the single embedding using the symbolic return embedding and the geometric return embedding. The geometric attention block 124 may generate the symbolic return embedding oᵢ for amino acid i, e.g., as:

$${o}_{i} = {\sum }_{j} {a}_{j} {v}_{j} \qquad ( 18 )$$

where [aⱼ]ⱼ₌₁^{N} denote the attention weights (e.g., defined with reference to equation (17)) and each vⱼ denotes the symbolic value embedding for amino acid j. The geometric attention block 810 may generate the geometric return embedding oᵢ^{p} for amino acid i, e.g., as:

$${o}_{i}^{p} = {R}_{i}^{- 1} \cdot ( {\sum }_{j} {a}_{j} {v}_{j}^{p} - {t}_{i} ) \qquad ( 19 )$$

where the geometric return embedding oᵢ^{p} is a 3-D point, [aⱼ]ⱼ₌₁^{N} denote the attention weights (e.g., defined with reference to equation (17)), Rᵢ⁻¹ is inverse of the rotation matrix specified by the rotation parameters for amino acid i, and tᵢ are the location parameters for amino acid i. It can be appreciated that the geometric return embedding is initially generated in the global reference frame, and then rotated and translated to the local reference frame of the corresponding amino acid.

The geometric attention block 810 may update the single embedding hᵢ for amino acid i using the corresponding symbolic return embedding oᵢ (e.g., generated in accordance with equation (18)) and geometric return embedding oᵢ^{p} (e.g., generated in accordance with equation (19)), e.g., as:

hᵢ^{next}=LayerNorm(hᵢ+Linear(oᵢ,oᵢ^{p},|oᵢ^{p}|)) (20)

where hᵢ^{next} is the updated single embedding for amino acid i, |⋅| is a norm, e.g., an L₂ norm, and LayerNorm(⋅) denotes a layer normalization operation, e.g., as described with reference to: J. L. Ba, J. R. Kiros, G. E. Hinton, “Layer Normalization,” arXiv:1607.06450 (2016).

Updating the single embeddings 806 of the amino acids using concrete 3-D geometric embeddings, e.g., as described with reference to equations (14)-(16), enables the geometric attention block 810 to reason about 3-D geometry in updating the single embeddings. Moreover, each update block updates the single embeddings and the structure parameters in a manner that is invariant to rotations and translations of the overall protein structure. For example, applying the same global rotation and translation operation to the initial structure parameters provided to the folding neural network 800 would cause the folding neural network 800 to generate a predicted structure that is globally rotated and translated in the same way, but otherwise the same. Therefore, global rotation and translation operations applied to the initial structure parameters would not affect the accuracy of the predicted protein structure generated by the folding neural network 800 starting from the initial structure parameters. The rotational and translational invariance of the representations generated by the folding neural network 800 facilitates training, e.g., because the folding neural network 800 automatically learns to generalize across all rotations and translations of protein structures.

The updated single embeddings for the amino acids may be further transformed by one or more additional neural network layers in the geometric attention block 810, e.g., linear neural network layers, before being provided to the folding block 812.

After the geometric attention block 810 updates the current single embeddings 806 for the amino acids, the folding block 812 updates the current structure parameters 808 using the updated single embeddings 816. For example, the folding block 812 may update the current location parameters tᵢ for amino acid i as:

tᵢ^{next}=tᵢ+Linear(hᵢ^{next}) (21)

where tᵢ^{next} are the updated location parameters, Linear(⋅) denotes a linear neural network layer, and hᵢ^{next} denotes the updated single embedding for amino acid i. In another example, the rotation parameters Rᵢ for amino acid i may specify a rotation matrix, and the folding block 812 may update the current rotation parameters Rᵢ as:

Wᵢ=Linear(hᵢ^{next}) (22)

Rᵢ^{next}=Rᵢ·QuaternionToRotation(1+wᵢ) (23)

where wᵢ is a three-dimensional vector, Linear(⋅) is a linear neural network layer, hᵢ^{next} is the updated single embedding for amino acid i, 1+wᵢ denotes a quaternion with real part 1 and imaginary part wᵢ, and QuaternionToRotation(⋅) denotes an operation that transforms a quaternion into an equivalent 3×3 rotation matrix. Updating the rotation parameters using equations (22)-(23) ensures that the updated rotation parameters define a valid rotation matrix, e.g., an orthonormal matrix with determinant one.

The folding neural network 800 may provide the updated structure parameters generated by the final update block 814 as the final structure parameters 314 that define the predicted protein structure 316. The folding neural network 800 may include any appropriate number of update blocks, e.g., 5 update blocks, 25 update blocks, or 125 update blocks. Optionally, each of the update blocks of the folding neural network may share a single set of parameter values that are jointly updated during training of the folding neural network. Sharing parameter values between the update blocks 814 reduces the number of trainable parameters of the folding neural network and may therefore facilitate effective training of the folding neural network, e.g., by stabilizing the training and reducing the likelihood of overfitting.

During training, a training engine can train the parameters of the structure prediction system, including the parameters of the folding neural network 800, based on a structure loss that evaluates the accuracy of the final structure parameters 314, as described above. In some implementations, the training engine can further evaluate an auxiliary structure loss for one or more of the update blocks 814 that precede the final update block (i.e., that produces the final structure parameters). The auxiliary structure loss for an update block evaluates the accuracy of the updated structure parameters generated by the update block.

Optionally, during training, the training engine can apply a “stop gradient” operation to prevent gradients from backpropagating through certain neural network parameters of each update block, e.g., the neural network parameters used to compute the updated rotation parameters (as described in equations (22)-(23)). Applying these stop gradient operations can improve the numerical stability of the gradients computed during training.

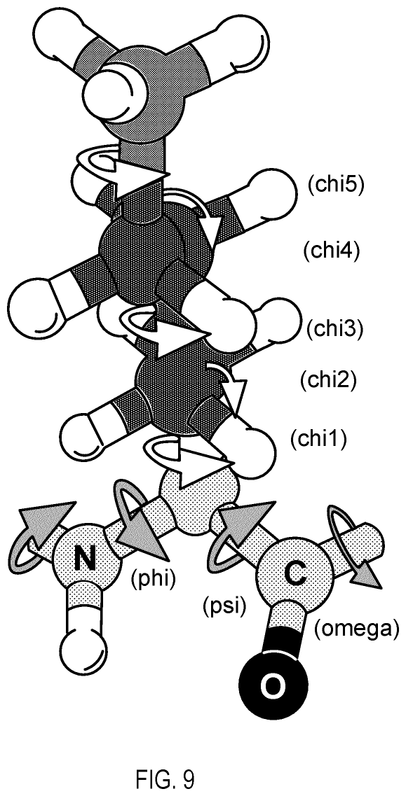

The location and rotation parameters specified by the structure parameters 314 can define the spatial locations (e.g., in [x,y,z] Cartesian coordinates) of the main chain atoms in the amino acids of the protein. However, the structure parameters 314 do not necessarily define the spatial locations of the remaining atoms in the amino acids of the protein, e.g., the atoms in the side chains of the amino acids. In particular, the spatial locations of the remaining atoms in an amino acid depend on the values of the torsion angles between the bonds in the amino acid, e.g., the omega-angle, the phi-angle, the psi-angle, the chi1-angle, the chi2-angle, the chi3-angle, and the chi4 angle, as illustrated with reference to FIG. 9.

Optionally, one or more of the update blocks 814 of the folding neural network 800 can generate an output that defines a respective predicted spatial location for each atom in each amino acid of the protein. To generate the predicted spatial locations for the atoms in an amino acid, the update block can process the updated single embedding for the amino acid using one or more neural network layers to generate predicted values of the torsion angles of the bonds between the atoms in the amino acid. The neural network layers may be, e.g., fully-connected neural network layers embedded with residual connections. Each torsion angle may be represented, e.g., as a 2-D vector.

The update block can determine the spatial locations of the atoms in an amino acid based on: (i) the values of the torsion angles for the amino acid, and (ii) the updated structure parameters (e.g., location and rotation parameters) for the amino acid. For example, the update block can process the torsion angles in accordance with a predefined function to generate the spatial locations of the atoms in the amino acid in a local reference frame of the amino acid. The update block can generate the spatial locations of the atoms in the amino acid in a global reference frame (i.e., that is common to all the amino acids in the protein) by rotating and translating the spatial locations of the atoms in accordance with the updated structure parameters for the amino acid. For example, the update block can determine the spatial location of an atom in the global reference frame by applying the rotation operation defined by the updated rotation parameters to the spatial location of the atom in the local reference frame to generate a rotated spatial location, and then apply the translation operation defined by the updated location parameters to the rotated spatial location.

In some implementations, alternatively to or in combination with outputting the final structure parameters, the folding neural network 800 outputs the predicted spatial locations of the atoms in the amino acids of the protein that are generated by the final update block.

The folding neural network 800 described with reference to FIG. 8 is characterized herein as receiving an input that is based on an MSA representation 310 and pair embeddings 312 that are generated by an embedding neural network, e.g., as described with reference to FIG. 4. In general, however, the inputs to the folding neural network (e.g., the single embeddings 802 and the pair embeddings 312) can be generated using any appropriate technique. Moreover, various aspects of the operations performed by the folding neural network (e.g., predicting spatial locations for the atoms in each amino acid of the protein) can be performed by other folding neural networks, e.g., with different architectures that receive different inputs.

FIG. 9 illustrates the torsion angles between the bonds in the amino acid, e.g., the omega-angle, the phi-angle, the psi-angle, the chit-angle, the chi2-angle, the chi3-angle, the chi4 angle, and the chi5 angle.

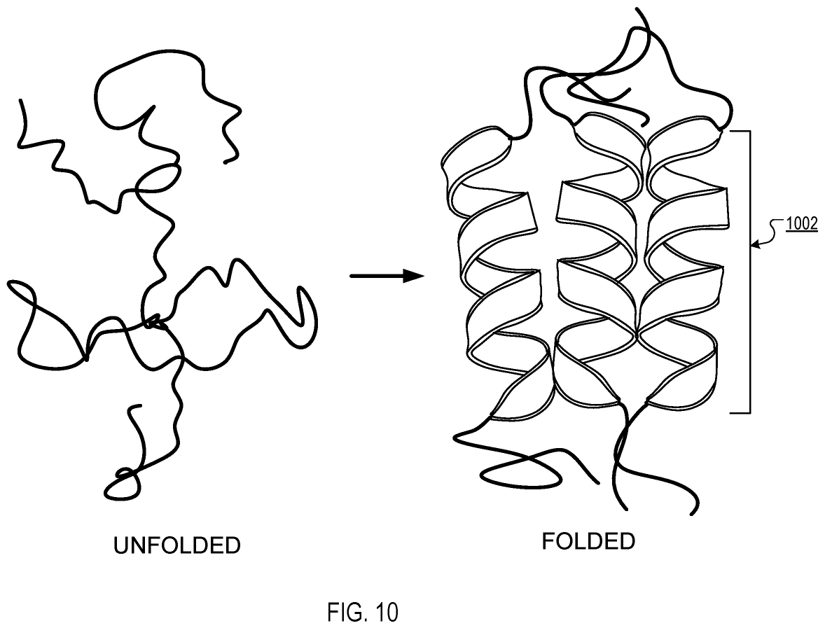

FIG. 10 is an illustration of an unfolded protein and a folded protein. The unfolded protein is a random coil of amino acids. The unfolded protein undergoes protein folding and folds into a 3D configuration. Protein structures often include stable local folding patterns such alpha helices (e.g., as depicted by 1002) and beta sheets.

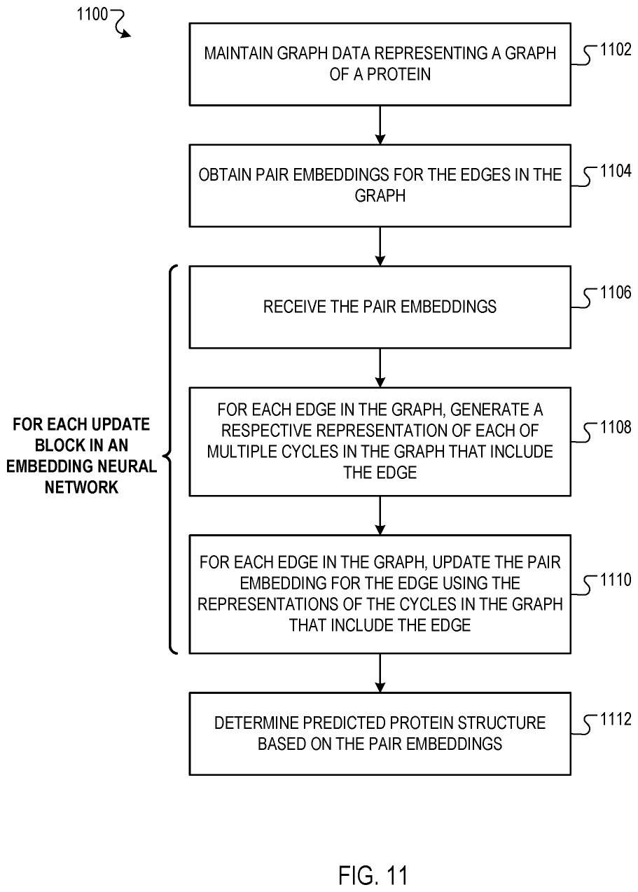

FIG. 11 is a flow diagram of an example process 1100 determining a predicted structure of a protein. For convenience, the process 1100 will be described as being performed by a system of one or more computers located in one or more locations. For example, a protein structure prediction system, e.g., the protein structure prediction system 300 of FIG. 3, appropriately programmed in accordance with this specification, can perform the process 1100.

The system maintains graph data representing a graph of the protein (1102). The graph includes a set of nodes and a set of edges. The set of nodes includes multiple amino acid nodes that each represent a respective amino acid in the protein. The set of edges includes a respective edge connecting each pair of amino acid nodes in graph.

The system obtains a respective pair embedding for each edge in the graph (1104). In some implementations, the graph data representing the graph of the protein can refer to an arrangement of the pair embeddings into an N×N array, where N is the number of amino acids in the protein, and position (i,j) in the array is occupied by the pair embedding corresponding to amino acid i and amino acid j in the protein. The number of rows/columns in the array defines the number of nodes in the graph, and the pair embedding at each position (i,j) in the array defines that: (i) node i and node j in the graph are connected by an edge, and the edge is associated with the pair embedding at position (i,j) in the array.

The system processes an input that includes the pair embeddings using an embedding neural network. The embedding neural network includes a sequence of update blocks and uses the update blocks to repeatedly update the pair embeddings. Steps 1206-1210, which are described next, are performed by each update block in the sequence of update blocks.

The update block receives the pair embeddings (1106).

For each edge in the graph that connects a pair of amino acid nodes, the update block generates a respective representation of each of multiple cycles in the graph that include the edge (1108). To generate a representation of a cycle in the graph, the update block processes embeddings for edges in the cycle in accordance with the values of the update block parameters.

For each edge in the graph that connects a pair of amino acid nodes, the update block updates the pair embedding for the edge using the representations of the cycles in the graph that include the edge (1110).

After processing the pair embeddings using the embedding neural network, the system determines the predicted structure of the protein based on the pair embeddings (1112).

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more data processing apparatus for determining a predicted structure of a protein, the method comprising:
maintaining graph data representing a graph of the protein, wherein the graph comprises a set of nodes and a set of edges, wherein the set of nodes comprises a plurality of amino acid nodes that each represent a respective amino acid in the protein, and wherein the set of edges comprises a respective edge connecting each pair of amino acid nodes in graph;
obtaining a respective pair embedding for each edge in the graph that connects a pair of amino acid nodes;
processing an input comprising the pair embeddings using an embedding neural network, wherein the embedding neural network comprises a sequence of update blocks and uses the update blocks to repeatedly update the pair embeddings,
wherein each update block has a plurality of update block parameters and performs operations comprising:
receiving the pair embeddings;
updating the pair embeddings in accordance with values of the update block parameters of the update block, comprising, for each edge in the graph that connects a pair of amino acid nodes:
generating a respective representation of each of a plurality of cycles in the graph that include the edge by, for each cycle, processing embeddings for edges in the cycle in accordance with the values of the update block parameters of the update block to generate the representation of the cycle; and
updating the pair embedding for the edge using the representations of the cycles in the graph that include the edge; and
after processing the pair embeddings using the embedding neural network, determining the predicted structure of the protein based on the pair embeddings.

2. The method of claim 1, wherein generating a respective representation of each of a plurality of cycles in the graph that include the edge comprises generating a respective representation of every cycle in the graph that includes the edge and that has a predefined length.

3. The method of claim 2, wherein the predefined length is three.

4. The method of claim 1, wherein updating the pair embedding for the edge using the representations of the cycles in the graph that include the edge comprises:
processing the pair embedding for the edge and the representations of the cycles in the graph that include the edge, in accordance with the values of the update block parameters of the update block, to generate a residual embedding; and
adding the residual embedding to the pair embedding for the edge.

5. The method of claim 4, wherein processing the pair embedding for the edge and the representations of the cycles in the graph that include the edge, in accordance with the values of the update block parameters of the update block, to generate a residual embedding comprises:
summing the representations of the cycles in the graph that include the edge; and
processing the pair embedding for the edge and the sum of the representations of the cycles in the graph that include the edge using one or more neural network layers to generate the residual embedding.

6. The method of claim 1, wherein each update block receives a multiple sequence alignment (MSA) representation for the protein that represents a respective MSA corresponding to each amino acid chain in the protein; and
wherein the pair embedding for each edge is updated based at least in part on the MSA representation.

7. The method of claim 6, wherein set of nodes of the graph comprises a plurality of multiple sequence alignment (MSA) sequence nodes that each represent a respective MSA sequence corresponding to an amino acid chain in the protein,
wherein the set of edges of the graph comprises a respective edge connecting each MSA sequence node in the graph to each amino acid node in the graph, and
wherein the MSA representation for the protein comprises a respective embedding for each edge in the graph that connects a MSA sequence node to an amino acid node.

8. The method of claim 7, wherein for each edge in the graph that connects a pair of amino acid nodes, generating the respective representation of each of the plurality of cycles in the graph that include the edge comprises generating a respective representation of each of one or more cycles in the graph that include an edge that connects a MSA sequence node to an amino acid node.

9. The method of claim 6, wherein each update block further performs operations comprising:
applying a transformation operation to the MSA representation; and
updating the pair embeddings by adding a result of the transformation operation to the pair embeddings.

10. The method of claim 9, wherein the transformation operation comprises an outer product mean operation.

11. The method of claim 6, wherein each update block further performs operations comprising:
updating the MSA representation based on the pair embeddings.

12. The method of claim 11, wherein updating the MSA representation based on the pair embeddings comprises:
updating the MSA representation using attention over embeddings in the MSA representation, wherein the attention is conditioned on the pair embeddings.

13. The method of claim 12, wherein updating the MSA representation using attention over the embeddings in the MSA representation comprises:
generating, based on the MSA representation, a plurality of attention weights;
generating, based on the pair embeddings, a respective attention bias corresponding to each of the attention weights;
generating a plurality of biased attention weights based on the attention weights and the attention biases; and
updating the embeddings in the MSA representation using attention over the embeddings in the MSA representation based on the biased attention weights.

14. The method of claim 13, wherein updating the embeddings in the MSA representation using attention based on the biased attention weights comprises, for each embedding in the MSA representation:
updating the embedding, based on the biased attention weights, using attention over only embeddings in the MSA representation that are located in a same row as the embedding in an arrangement of the embeddings in the MSA representation into a two-dimensional array.

15. A system comprising:
one or more computers; and
one or more storage devices communicatively coupled to the one or more computers, wherein the one or more storage devices store instructions that, when executed by the one or more computers, cause the one or more computers to perform operations for determining a predicted structure of a protein, the operations comprising:
maintaining graph data representing a graph of the protein, wherein the graph comprises a set of nodes and a set of edges, wherein the set of nodes comprises a plurality of amino acid nodes that each represent a respective amino acid in the protein, and wherein the set of edges comprises a respective edge connecting each pair of amino acid nodes in graph;
obtaining a respective pair embedding for each edge in the graph that connects a pair of amino acid nodes;
processing an input comprising the pair embeddings using an embedding neural network, wherein the embedding neural network comprises a sequence of update blocks and uses the update blocks to repeatedly update the pair embeddings,
wherein each update block has a plurality of update block parameters and performs operations comprising:
receiving the pair embeddings;
updating the pair embeddings in accordance with values of the update block parameters of the update block, comprising, for each edge in the graph that connects a pair of amino acid nodes:
generating a respective representation of each of a plurality of cycles in the graph that include the edge by, for each cycle, processing embeddings for edges in the cycle in accordance with the values of the update block parameters of the update block to generate the representation of the cycle; and
updating the pair embedding for the edge using the representations of the cycles in the graph that include the edge; and
after processing the pair embeddings using the embedding neural network, determining the predicted structure of the protein based on the pair embeddings.

16. One or more non-transitory computer storage media storing instructions that when executed by one or more computers cause the one or more computers to perform operations for determining a predicted structure of a protein, the operations comprising:
maintaining graph data representing a graph of the protein, wherein the graph comprises a set of nodes and a set of edges, wherein the set of nodes comprises a plurality of amino acid nodes that each represent a respective amino acid in the protein, and wherein the set of edges comprises a respective edge connecting each pair of amino acid nodes in graph;
obtaining a respective pair embedding for each edge in the graph that connects a pair of amino acid nodes;
processing an input comprising the pair embeddings using an embedding neural network, wherein the embedding neural network comprises a sequence of update blocks and uses the update blocks to repeatedly update the pair embeddings,
wherein each update block has a plurality of update block parameters and performs operations comprising:
receiving the pair embeddings;
updating the pair embeddings in accordance with values of the update block parameters of the update block, comprising, for each edge in the graph that connects a pair of amino acid nodes:
generating a respective representation of each of a plurality of cycles in the graph that include the edge by, for each cycle, processing embeddings for edges in the cycle in accordance with the values of the update block parameters of the update block to generate the representation of the cycle; and
updating the pair embedding for the edge using the representations of the cycles in the graph that include the edge; and
after processing the pair embeddings using the embedding neural network, determining the predicted structure of the protein based on the pair embeddings.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. The non-transitory computer storage media of claim 16, wherein generating a respective representation of each of a plurality of cycles in the graph that include the edge comprises generating a respective representation of every cycle in the graph that includes the edge and that has a predefined length.

29. The non-transitory computer storage media of claim 28, wherein the predefined length is three.

30. The non-transitory computer storage media of claim 16, wherein updating the pair embedding for the edge using the representations of the cycles in the graph that include the edge comprises:
processing the pair embedding for the edge and the representations of the cycles in the graph that include the edge, in accordance with the values of the update block parameters of the update block, to generate a residual embedding; and
adding the residual embedding to the pair embedding for the edge.

31. The non-transitory computer storage media of claim 30, wherein processing the pair embedding for the edge and the representations of the cycles in the graph that include the edge, in accordance with the values of the update block parameters of the update block, to generate a residual embedding comprises:
summing the representations of the cycles in the graph that include the edge; and
processing the pair embedding for the edge and the sum of the representations of the cycles in the graph that include the edge using one or more neural network layers to generate the residual embedding.
